SEQUENCE LISTING (14 sequences)

SEQ ID NO: 1 - DNA, 20 bp, Artificial Sequence
  TNF-alpha forward
ctcttctcattcccgctcgt

SEQ ID NO: 2 - DNA, 20 bp, Artificial Sequence
  TNF-alpha reverse
gggagcccatttgggaactt

SEQ ID NO: 3 - DNA, 20 bp, Artificial Sequence
  IL-6 Forward
ccagttgccttcttgggact

SEQ ID NO: 4 - DNA, 20 bp, Artificial Sequence
  IL-6 Reverse
tctgacagtgcatcatcgct

SEQ ID NO: 5 - DNA, 19 bp, Artificial Sequence
  ICAM-1 forward
caaacgggagatgaatggt

SEQ ID NO: 6 - DNA, 21 bp, Artificial Sequence
  ICAM-1 Reverse
tctggcggtaataggtgtaaa

SEQ ID NO: 7 - DNA, 19 bp, Artificial Sequence
  MCP-1 Forward
ctgaccccaataaggaatg

SEQ ID NO: 8 - DNA, 20 bp, Artificial Sequence
  MCP-1 Reverse
tgaggtggttgtggaaaaga

SEQ ID NO: 9 - DNA, 25 bp, Artificial Sequence
  VCAM-1 Forward
tttgcaagaaaagccaacatgaaag

SEQ ID NO: 10 - DNA, 23 bp, Artificial Sequence
  VCAM-1 Reverse
tctccaacagttcagacgttagc

SEQ ID NO: 11 - DNA, 21 bp, Artificial Sequence
  NF-kB forward
acgatctgtttcccctcatct

SEQ ID NO: 12 - DNA, 21 bp, Artificial Sequence
  NB-kB Reverse
tgggtgcgtcttagtggtatc

SEQ ID NO: 13 - DNA, 20 bp, Artificial Sequence
  GAPDH Forward
aaacccatcaccatcttcca

SEQ ID NO: 14 - DNA, 20 bp, Artificial Sequence
  GAPDH Reverse
gtggttcacacccatcacaa

(19) United States
(12) Patent Application Publication
(10) Pub. No.: US 2023/0065014 A1
(43) Pub. Date: Mar. 2, 2023

(54) Nitro-Fatty Acid-Containing Microbubbles and Uses Therefor

(71) Applicant: University of Pittsburgh-Of the Commonwealth System of Higher Education, Pittsburgh, PA (US)

(72) Inventors: Gary Yu, Pittsburgh, PA (US); Bruce A. Freeman, Pittsburgh, PA (US); Flordeliza S. Villanueva, Pittsburgh, PA (US); John J. Pacella, Bethel Park, PA (US); Marco Fazzari, Pittsburgh, PA (US); Xucai Chen, Pittsburgh, PA (US); Thiruganesh Ramasamy, San Francisco, CA (US)

(21) Appl. No.: 17/792,653

(22) PCT Filed: Jan. 14, 2021
(86) PCT No.: PCT/US2021/013446

§ 371 Date: Jul. 13, 2022

Related U.S. Application Data
(60) Provisional application No. 62/960,923, filed on Jan. 14, 2020.

(51) Int. Cl. A61K 9/50; A61K 47/10; A61K 47/24; A61K 31/19; A61P 9/10

(52) U.S. Cl. CPC A61K 9/5015; A61K 47/10; A61K 47/24; A61K 31/19; A61P 9/10

(57) ABSTRACT

Provided herein are microbubble compositions comprising nitro-fatty acids and/or esters thereof, such as amphiphilic esters or allyl esters thereof. Also provided are methods of reducing local inflammation at a site in a patient comprising delivering the microbubbles to a site of inflammation in the patient and applying ultrasound to the microbubbles. The methods may be used to treat fibrosis or cancer.

### CROSS REFERENCE TO RELATED APPLICATIONS

This application claims the benefit of co-pending U.S. Provisional Application, No. 62/960,923, filed Jan. 14, 2020, which is incorporated herein by reference in its entirety.

### STATEMENT REGARDING FEDERAL FUNDING

This invention was made with government support under Grant Nos. HL125777, HL64937, HL132550, and HL103455 awarded by the National Institutes of Health. The government has certain rights in the invention.

### REFERENCE TO A SEQUENCE LISTING SUBMITTED VIA EFS-WEB

The content of the ASCII text file of the sequence listing named “6527_2006602_ST25” which is 3,061 bytes in size was created on Jan. 14, 2021 and electronically submitted herewith via EFS-Web, and is hereby incorporated by reference in its entirety.

Microvascular obstruction (MVO) is a highly prevalent problem following percutaneous coronary intervention (PCI) for the treatment of acute myocardial infarctions and leads to worse clinical outcomes. MVO arises from a variety of mechanisms including downstream micro-embolization resulting in mechanical obstruction, myocardial edema, inflammation, and a milieu of oxidative stress accompanying ischemia-reperfusion injury. MVO occurs in up to 60% of all ST-Elevation Myocardial Infarction (STEM) heart attack patients receiving PCI, causing decreased left ventricular (LV) function and independently predicting major adverse cardiac events (cardiac death, stroke, myocardial infarction, and heart failure requiring hospitalization). MVO is linked with increased infarct size and negative LV remodeling, with MVO persistence a stronger predictor for functional recovery than transmural infarct extension.

Nitro-fatty acids (NFAs) are naturally occurring nitrated fatty acids that have been shown to have a wide range of health benefits when given therapeutically. These include decreasing oxidative stress, inflammation, and improving nitric oxide bioavailability. In addition, NFAs modulate over 500 genes via post-translation modification of regulatory proteins that affect oxidative stress, inflammation, and hypoxic response. NFAs have net therapeutic benefits of decreased production of oxidative inflammatory mediators, inhibition of platelet and white blood cell activation, inhibition of pathogenic remodeling (fibrosis), preservation of normal endothelial function, and cardioprotection in the setting of focal and global injury. NFAs can be safely administered in humans both orally and intravenously and have been explored in a wide range of cardiovascular disease contexts including atherosclerosis, myocardial infarction, hypertension, atrial fibrillation, and more.

NFAs are currently administered in a systemic fashion with no targeted specificity to the location of pathology. Many investigated pathologies, such as myocardial infarction, are highly localized and would benefit from such capacity. In addition, activity of NFAs has been shown to be both dose- and time-dependent. Given their relatively rapid onset of effectiveness and lack of adverse effects from overdosing, NFAs are a strong candidate for targeted therapy in the context of MVO.

There is a need in the art for the targeted-localized delivery of NFAs for immediate concentration at tissue specific sites to enhance therapeutic efficacy.

### SUMMARY

A microbubble composition is provided, comprising: a lipid-based shell comprising a circumferential region that defines a core of the microbubble, a gas within the core of the microbubble, and a nitro-fatty acid or an amphiphilic ester thereof, or a pharmaceutically-acceptable salt of the nitro-fatty acid or amphiphilic ester thereof.

A therapeutic composition is provided, comprising the microbubble; and a pharmaceutically acceptable excipient, one or more additional therapeutically active agents or visualization agents, or any combinations thereof. A unit dosage form (e.g., injectable) is provided, comprising the therapeutic composition in a medical syringe or a reservoir configured to connect to a catheter, such as an infusion catheter or a catheter configured for use in a thrombolysis procedure.

A method of treating localized inflammation in a patient is provided, comprising delivering the microbubble composition as described in the preceding paragraph, to a site of localized inflammation in the patient and administering an ultrasound pulse to the site effective to deliver the nitro-fatty acid to the site or to disrupt the microbubble, thereby reducing inflammation at the site.

A method of clot thrombolysis in a patient is provided, comprising delivering the microbubble composition to a site of a thrombus or microvascular obstruction in a patient, and administering ultrasound effective for thrombolysis of the thrombus or the microvascular obstruction and to release the nitro-fatty acid at the site.

A method of treating a fibrotic lesion in a patient is provided, comprising delivering the microbubble composition to a site of a fibrotic lesion in a patient, and administering ultrasound effective to release the nitro-fatty acid at the site, thereby reducing the size of fibrotic lesion and/or preventing further fibrosis.

A method of reducing inflammation in a patient is provided, comprising delivering a gas-filled microbubble and a nitro-fatty acid or an amphiphilic ester thereof, or a pharmaceutically-acceptable salt of the nitro-fatty acid or amphiphilic ester thereof, to a site of localized inflammation in a patient and administering an ultrasound pulse to the site effective to disrupt the microbubbles, thereby reducing inflammation at the site.

The following numbered clauses outline various non-limiting aspects, embodiments, and/or examples of the present invention.

1. A microbubble, comprising: a lipid-based shell comprising a circumferential region that defines a core of the microbubble, a gas within the core of the microbubble, and a nitro-fatty acid or an amphiphilic ester thereof, or a pharmaceutically-acceptable salt of the nitro-fatty acid or amphiphilic ester thereof.

2. The microbubble of clause 1, wherein the nitro-fatty acid or amphiphilic ester thereof, or a pharmaceutically-acceptable salt of the nitro-fatty acid or amphiphilic ester thereof is incorporated into the exterior circumferential region of the lipid-based shell.

3. The microbubble of clause 1 or 2, wherein the nitro-fatty acid or amphiphilic ester thereof, or a pharmaceutically-acceptable salt of the nitro-fatty acid or amphiphilic ester thereof is contained within the core of the microbubble.

4. The microbubble of any one of clauses 1-4, wherein the gas comprises air, oxygen, nitrogen, argon, or a perfluorocarbon.

5. The microbubble of clause 4, wherein the perfluorocarbon is perfluorobutane.

6. The microbubble of any one of clauses 1-5, wherein the lipid based shell comprises a phospholipid.

7. The microbubble of any one of clauses 1-6, wherein the lipid-based shell comprises a phosphotidylcholine.

8. The microbubble of any one of clauses 1-7, wherein the lipid-based shell comprises a glycerol-phosphoethanolamine lipid that is optionally PEGylated.

9. The microbubble of any one of clauses 1-8, wherein the lipid-based shell comprises a PEGylated fatty acid.

10. The microbubble of any one of clauses 1-5, wherein the lipid-based shell comprises a phospholipid, a phosphotidylcholine, a glycerol-phosphoethanolamine lipid, and a PEGylated fatty acid.

11. The microbubble of any one of clauses 1-10, wherein the lipid-based shell comprises:

a. 1,2-distearoyl-sn-glycero-3-phosphocholine, e.g.,

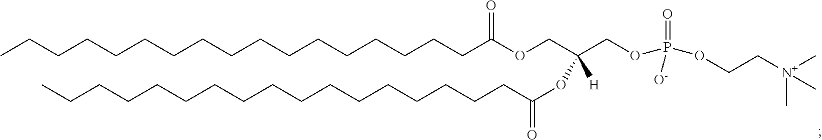 [SMILES: CCCCCCCCCCCCCCCCCC(=O)OC[C@H](COP(=O)([O-])OCC[N+](C)(C)C)OC(=O)CCCCCCCCCCCCCCCCC]

b. 1,2-Distearoyl-sn-Glycero-3-Phosphoethanolamine-N-[Methoxy(Polyethylene glycol)-2000], e.g.,

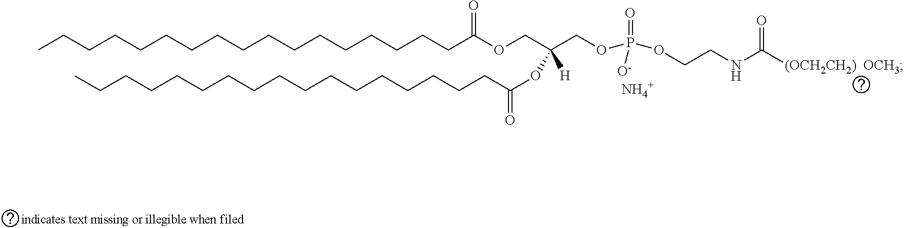

and

c. PEG-stearate, e.g. polyoxyethylene (40) stearate, e.g.,

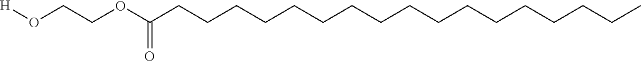 [SMILES: CCCCCCCCCCCCCCCCCC(=O)OCCO]

12. The microbubble of any one of clauses 1-11, where the nitro-fatty acid or amphiphilic ester thereof, or pharmaceutically-acceptable salt of the nitro-fatty acid or amphiphilic ester thereof, is associated with the lipid-based shell in a ratio as compared to other lipids present in the lipid shell ranging from 10 to 25% by mass or from 20 to 40% by moles, from 7.5 to 17.5% by mass or from 15 to 30% by moles, from 10 to 15% by mass or from 18 to 25% by moles, about 12.5% by mass and about 21.9% by moles, or 12.5% by mass and 21.9% by moles.

13. The microbubble of any one of clauses 1-12, comprising a nitro-fatty acid.

14. The microbubble of clause 13, wherein the nitro-fatty acid is: 9-nitrooleic acid, 10-nitrooleic acid, 9-nitrolinoleic acid, 10-nitrolinoleic acid, 12-nitrolinoleic acid, 13-nitrolinoleic acid, 9-Nitrononanoic acid, 9-nitro-9-trans-octadecenoic acid, 10-nitro-9-trans-octadecenoic acid, or a salt or ester thereof, e.g., an allyl ester or an amphiphilic ester thereof, such as a phospholipid.

15. The microbubble of any one of clauses 1-14, comprising a nitro-fatty acid having the structure:

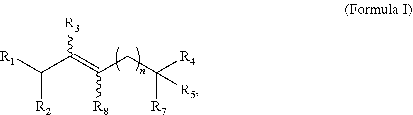

wherein R₁ is hydrogen, C₁-C₂₄ alkyl or C₁-C₂₄ alkenyl; R₂, R₃, R₇, and R₈ are each independently, hydrogen, oxygen, C₁-C₂₄ alkyl or NO₂; R₄ is a terminal COOR₆ group, wherein R₆ is hydrogen, C₁-C₂₄ alkyl, or a pharmaceutically acceptable counterion; R₅ is hydrogen, C₁-C₂₄ alkyl, or R₄ and R₅ collectively form ═C(R₉)(R₁₀), wherein R₉ comprises C₁-C₂₄ alkyl or C₁-C₂₄ alkenyl, or wherein R₉ is a terminal COOR₆ group, and R₁₀ is hydrogen or NO₂; n is from 1 to 24; and wherein at least one of R₂, R₃, R₇, R₈, and R₁₀, when present, is an NO₂ group;

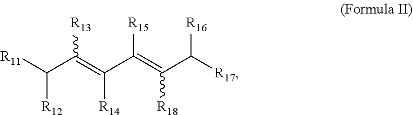

wherein R₁₁ is hydrogen, C₁-C₂₄ alkyl, or C₁-C₂₄ alkenyl; R₁₂, R₁₄, R₁₅ and R₁₆ are hydrogen, C₁-C₂₄ alkyl, or C₁-C₂₄ alkenyl; R₁₃ and R₁₈ are each independently, hydrogen, oxygen, C₁-C₂₄ alkyl, or NO₂; R₁₇ is a terminal COOR₁₉ group, wherein R₁₉ is hydrogen, C₁-C₂₄ alkyl, or a pharmaceutically acceptable counterion, wherein at least one of R₁₃, R₁₄, R₁₅, and R₁₈ is NO₂; or

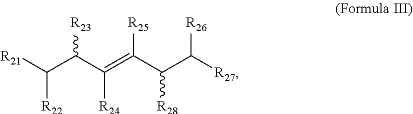

wherein R₂₁ is hydrogen, C₁-C₂₄ alkyl, or C₁-C₂₄ alkenyl; R₂₂, R₂₄, R₂₅′ and R₂₆ are hydrogen; one of R₂₃ and R₂₈ is NO₂, and the other of R₂₃ and R₂₈ is ONO₂; and R₂₇ is a terminal COOR₂₉ group, wherein R₂₉ is hydrogen, C₁-C₂₄ alkyl, or a pharmaceutically acceptable counterion,
or an amphiphilic ester thereof, optionally a phospholipid or phosphoglyceride thereof, or a pharmaceutically-acceptable salt of the nitro-fatty acid or amphiphilic ester thereof.

16. A therapeutic composition, comprising: a microbubble of any one of clauses 1-15; and a pharmaceutically acceptable excipient, one or more additional therapeutically active agents or visualization agents, or any combinations thereof.

17. A unit dosage form (e.g., injectable) comprising the therapeutic composition of clause 16 in a medical syringe or a reservoir configured to connect to a catheter, such as an infusion catheter or a catheter configured for use in a thrombolysis procedure.

18. A method of treating localized inflammation in a patient, comprising delivering the composition of clause 16, to a site of localized inflammation in the patient and administering an ultrasound pulse to the site effective to deliver the nitro-fatty acid or amphiphilic ester thereof, or the pharmaceutically-acceptable salt of the nitro-fatty acid or amphiphilic ester thereof, to the site or to disrupt the microbubble, thereby reducing inflammation at the site.

19. The method of clause 18, wherein the site of localized inflammation is associated with a thrombus or a microvascular obstruction and the ultrasound pulse is effective for thrombolysis of the thrombus or the microvascular obstruction.

20. The method of clause 18, wherein the site of localized inflammation is associated with fibrosis or cancer.

21. The method of clause 18, wherein the site of localized inflammation is a site of a stent, an aneurism coil, or a valve replacement.

22. The method of clause 18, wherein the site of localized inflammation is a site of a cardiovascular surgical procedure.

23. The method of clause 18, wherein the site of localized inflammation is a site of a pulmonary embolism.

24. The method of clause 18, wherein the site of localized inflammation is a site of a fibrotic lesion.

25. The method of clause 18, wherein the site of localized inflammation is a site of a tumor or precancerous lesion.

26. A method of clot thrombolysis in a patient, comprising delivering the composition of clause 16 to a site of a thrombus or microvascular obstruction in a patient, and administering ultrasound effective for thrombolysis of the thrombus or the microvascular obstruction and to release the nitro-fatty acid or amphiphilic ester thereof, or the pharmaceutically-acceptable salt of the nitro-fatty acid or amphiphilic ester thereof, at the site.

27. The method of clause 26, wherein the site of the thrombus or microvascular obstruction in the patient is in the patient's heart.

28. The method of clause 27, wherein the site of the thrombus or microvascular obstruction in the patient is in or adjacent to a myocardial infarct, thrombus, or microvascular obstruction.

29. A method of treating a fibrotic lesion in a patient, comprising delivering the composition of clause 16 to a site of a fibrotic lesion in a patient, and administering ultrasound effective to release the nitro-fatty acid or amphiphilic ester thereof, or the pharmaceutically-acceptable salt of the nitro-fatty acid or amphiphilic ester thereof, at the site, thereby reducing the size of fibrotic lesion and/or preventing further fibrosis.

30. A method of reducing inflammation in a patient, comprising delivering a gas-filled microbubble and a nitro-fatty acid or an amphiphilic ester thereof, or a pharmaceutically-acceptable salt of the nitro-fatty acid or amphiphilic ester thereof to a site of localized inflammation in a patient and administering an ultrasound pulse to the site effective to disrupt the microbubbles, thereby reducing inflammation at the site.

31. The method of clause 30, wherein the site of localized inflammation is associated with a thrombus or a microvascular obstruction and the ultrasound pulse is effective for thrombolysis of the thrombus or the microvascular obstruction.

32. The method of clause 30, wherein the site of localized inflammation is associated with fibrosis or cancer.

33. The method of clause 30, wherein the site of localized inflammation is a site of a stent, an aneurism coil, or a valve replacement.

34. The method of clause 30, wherein the site of localized inflammation is a site of a cardiovascular surgical procedure.

35. The method of clause 30, wherein the site of localized inflammation is a site of a pulmonary embolism.

36. The method of clause 30, wherein the site of localized inflammation is a site of a fibrotic lesion.

37. The method of clause 30, wherein the site of localized inflammation is a site of a tumor or precancerous lesion.

### BRIEF DESCRIPTION OF THE DRAWINGS

FIG. 1 is a diagram depicting the delivery of NFA from Nitro-fatty acid microbubbles (NFABs) using ultrasound-targeted microbubble cavitation (UTMC).

FIG. 2 is a graph depicting that the size distribution of NFAB was stable over time. Coulter counter measurements of NFAB size and concentration distributions over time were collected. Samples of NFABs from the same synthesis batch were serially sampled for measurement in a Coulter counter for distributions of microbubble counts over microbubble diameters, showing stability of the size and size distribution up to 4 days post-synthesis.

FIG. 3 is a graph depicting the quantification of loaded NFA for NFABs. The samples of NFABs in saline were quantified for NFA concentration using liquid chromatography/mass spectrometry methods. Subnatant samples with no NFABs were also quantified for concentration of free, unincorporated NFA which was then subtracted from total NFA concentration, resulting in the shown measurements.

FIG. 4 depicts sequential frames of high-speed video of NFABs stimulated by ultrasound (1 MegaHertz (MHz), 1.5 MegaPascal (MPa)) show typical lipid microbubble behaviors, including collapse and formation of daughter microbubbles. Frame size shown is 40 micrometers (μm).

FIG. 5 depicts burst-replenishment still-frame images from contrast-enhanced ultrasound imaging of healthy hindlimb model. The still-frame observational analysis at 5 seconds post-burst show drastic differences in replenishment between NFAB+UTMC and Free NFA+UTMC groups. Much of the microvascular bed is yet to be replenished in the latter, suggesting possible microvascular slowing or previously discussed microvascular spasm, while the former group shows full replenishment of the microvasculature exceeding baseline levels. TX1 for treatment 1, TX2 for treatment 2 imaging periods after a 10-minute treatment.

FIG. 6 is a graph depicting the changes in microvascular blood volume for the healthy hindlimb model. The changes in microvascular blood volume show similarly significant increases from baseline for both NFAB+UTMC and free NFA+UTMC groups compared against free NFA only. Results were analyzed using two-way ANOVA with post-hoc analysis performed using Sidak's multiple comparisons test. Asterisk indicates p<0.05 compared to both other groups. TX1 and TX2 indicate imaging was obtained after each respective 10-minute treatment, BL indicates baseline imaging prior to treatments.

FIG. 7 is a graph depicting the change in microvascular flow rate for healthy hindlimb model. Only the NFAB+UTMC group demonstrated significantly increased microvascular blood flow rate compared to free NFA only after both treatments. The NFAB+UTMC group also demonstrated significantly increased microvascular blood flow rate over the free NFA+UTMC group after treatment 2. Results were analyzed using two-way ANOVA with post-hoc analysis performed using Sidak's multiple comparisons test. Asterisk between bracketed groups indicates p<0.05. TX1 and TX2 indicate imaging was obtained after each respective 10-minute treatment, BL indicates baseline imaging prior to treatments.

FIG. 8 is a graph depicting change in tissue concentration of nitro-fatty acid. Change in NFA concentration was taken as a difference between ultrasound treated and untreated regions on the ipsilateral hindlimb on the same animal. All samples were treated as pairwise differences of treated minus untreated concentrations of NFA per mg protein. Differences for each group were analyzed using one-way ANOVA with post-hoc analysis using Tukey's multiple comparisons test. Asterisk indicates p<0.05 compared against both other groups. Bracketed p-value between the two UTMC-treated groups is as shown.

FIG. 9 depicts still-frame contrast-enhanced ultrasound images of ischemia-reperfusion injury model. Still-frame images were taken from burst-replenishment contrast-enhanced cine-loops at 5 seconds post-burst. Note the complete replenishment of microvasculature in the NFAB+UTMC group while the free NFA+UTMC group shows a decrease in microvascular perfusion.

FIG. 10 is a graph depicting changes in microvascular blood volume in the IR model. Significant increases in microvascular blood volume are only seen in the NFAB+UTMC group for the ischemia-reperfusion model after each treatment. Compared to the healthy hindlimb model, no significant increases in microvascular blood volume were seen in the free NFA+UTMC group after the post-ischemia-reperfusion imaging interval. Asterisk indicates p<0.05 for bracketed groups. TX1 for treatment 1, TX2 for treatment 2 imaging periods after a 10-minute treatment.

FIG. 11 is a graph depicting changes in microvascular blood flow rate in the ischemia-reperfusion model. Similar to microvascular blood volume, only the NFAB+UTMC group showed significant increases in microvascular blood flow rate compared to all other groups. Dagger indicates p<0.05 against all other groups. TX1 for treatment 1, TX2 for treatment 2 imaging periods after a 10-minute treatment.

FIGS. 12A-12F are graphs depicting Gene expression changes after NFAB-UTMC treatment. Changes in gene expression for key genes in inflammation and recruitment of leukocytes are shown for skeletal muscle tissue samples taken from control (no ultrasound treatment, “control”) and ultrasound treated sites on the ipsilateral hindlimb from the same animal. FIG. 12A depicts the percentage of relative I-CAM1 mRNA expression. FIG. 12B depicts the percentage of relative V-CAM1 mRNA expression. FIG. 12C depicts the percentage of relative IL-6 mRNA expression. FIG. 12D depicts the percentage of relative MCP mRNA expression.

FIG. 12E depicts the percentage of relative TNF-α mRNA expression. FIG. 12F depicts the percentage of relative NF-κB mRNA expression. Only the NFAB+UTMC group showed significant reductions in mRNA expression for the target genes in the treatment site compared to the other groups. All samples were normalized against a control group receiving ischemia-reperfusion injury only. Control and treatment sites were subjected to the same ischemia-reperfusion injury. Significance was calculated using a 2-way ANOVA with post-hoc comparison using Sidak's multiple comparisons test. Asterisk indicates p<0.05 between bracketed groups.

FIG. 13 is a graph depicting the measurement of MDA per mg protein for treatment site tissue samples, where NFAB+UTMC reduces lipid peroxidation. NFAB+UTMC resulted in the lowest concentration of MDA indicating the least extent of lipid peroxidation compared to either NFA alone or NFA+UTMC. All samples were normalized against a control group receiving ischemia-reperfusion injury only. Significance was calculated using a one-way ANOVA with post-hoc analysis using Tukey's multiple comparisons test. Asterisk indicates p<0.05 for the indicated group compared to both other groups.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect basic and novel characteristic(s). The term “consisting of” excludes any element, step, or ingredient not specified in the claim. As used herein, embodiments “comprising” one or more stated elements or steps also include, but are not limited to embodiments “consisting essentially of” and “consisting of” these stated elements or steps.

As used herein, the term “patient” or “subject” refers to members of the animal kingdom including but not limited to human beings and “mammal” refers to all mammals, including, but not limited to human beings.

A composition is “biocompatible” in that the composition and, where applicable, elements thereof, or degradation products thereof, are substantially non-toxic to cells or organisms within acceptable tolerances, including substantially non-carcinogenic and substantially non-immunogenic, and are cleared or otherwise degraded in a biological system, such as an organism (patient) without substantial toxic effect. Non-limiting examples of degradation mechanisms within a biological system include chemical reactions, hydrolysis reactions, and enzymatic cleavage.

As used herein, the terms “treating”, or “treatment” can refer to a beneficial or specific result, such as improving one of more functions, or symptoms of a disease. The terms “treating” or “treatment” can also include, but are not limited to, alleviation or amelioration of one or more symptoms of a repeat expansion disease, such as DM1, DM2, or Huntington's Disease. “Treatment” can also mean prolonging survival as compared to expected survival in the absence of treatment. As used herein, “percutaneous” refers to “through the skin.”

“Lower,” in the context of a disease marker or symptom, can refer to a clinically-relevant and/or a statistically significant decrease in such level. The decrease can be, for example, at least 10%, at least 20%, at least 30%, at least 40%, or more, down to a level accepted as within the range of normal for an individual without such disorder, or to below the level of detection of the assay. The decrease can be down to a level accepted as within the range of normal for an individual without such disorder, which can also be referred to as a normalization of a level. The reduction can be the normalization of the level of a sign or symptom of a disease, that is, a reduction in the difference between the subject level of a sign of the disease and the normal level of the sign for the disease (e.g., to the upper level of normal when the value for the subject must be decreased to reach a normal value, and to the lower level of normal when the value for the subject must be increased to reach a normal level). The methods may include a clinically relevant reduction of inflammation, microvascular obstructions, stenosis, restenosis, e.g., as demonstrated by a clinically relevant outcome after treatment of a subject with a NFA as described herein.

“Therapeutically effective amount,” as used herein, can include the amount of an NFA and microbubble as described herein that, when administered to a subject having a disease, can be sufficient to effect treatment of the disease (e.g., by diminishing, ameliorating or maintaining the existing disease or one or more symptoms of disease). The “therapeutically effective amount” may vary depending on the microbubble and NFA, how the composition is administered, the ultrasound treatment protocol, the disease and its severity and the history, age, weight, family history, genetic makeup, the types of preceding or concomitant treatments, if any, and other individual characteristics of the subject to be treated.

A “therapeutically-effective amount” can also include an amount of an agent that produces a local or systemic effect at a reasonable benefit/risk ratio applicable to any treatment. Microbubbles and NFAs employed in the methods described herein may be administered in a sufficient amount to produce a reasonable benefit/risk ratio applicable to such treatment.

The phrase “pharmaceutically-acceptable carrier” as used herein can refer to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier can be “acceptable” in the sense of being compatible with the other ingredients of the formulation and not injurious to the subject being treated. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (24) other non-toxic compatible substances employed in pharmaceutical formulations.

A “group” or “functional group” is a portion of a larger molecule comprising or consisting of a grouping of atoms and/or bonds that confer a chemical or physical quality to a molecule. A “residue” is the portion of a compound or monomer that remains in a larger molecule, such as a polymer chain, after incorporation of that compound or monomer into the larger molecule. A “moiety” is a portion of a molecule, and can comprise one or more functional groups, and in the case of an “active moiety” can be a characteristic portion of a molecule or compound that imparts activity, such as pharmacological or physiological activity, to a molecule as contrasted to inactive portions of a molecule such as esters of active moieties, or salts of active agents.

As used herein, “alkyl” refers to straight, branched chain, or cyclic hydrocarbon groups including, for example, from 1 to about 20 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. “lower alkyl” refers to C₁-C₆ alkyl. Non-limiting examples of straight alkyl groups include methyl, ethyl, propyl, butyl, pentyl, hexyl, heptyl, octyl, nonyl, and decyl. Branched alkyl groups comprises any straight alkyl group substituted with any number of alkyl groups. Non-limiting examples of branched alkyl groups include isopropyl, n-butyl, isobutyl, sec-butyl, and t-butyl. Non-limiting examples of cyclic alkyl groups include cyclopropyl, cyclobutyl, cyclopentyl, cyclohexyl, cycloheptlyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups. “Substituted alkyl” can include alkyl substituted at 1 or more (e.g., 1, 2, 3, 4, 5, 6, or more) positions, which substituents are attached at any available atom to produce a stable compound, with substitution as described herein. “Optionally substituted alkyl” refers to alkyl or substituted alkyl. “Halogen,” “halide,” and “halo” refers to —F, —Cl, —Br, and/or —I. “Alkylene” and “substituted alkylene” can include divalent alkyl and divalent substituted alkyl, respectively, including, without limitation, methylene, ethylene, trimethylene, tetramethylene, pentamethylene, hexamethylene, hepamethylene, octamethylene, nonamethylene, or decamethylene. “Optionally substituted alkylene” can include alkylene or substituted alkylene.

“Alkene or alkenyl” can include straight, branched chain, or cyclic hydrocarbyl groups including, e.g., from 2 to about 20 carbon atoms, such as, without limitation C₆₋₂₄ groups in the case of fatty acids, having one or more, e.g., 1, 2, 3, 4, or 5, carbon-to-carbon double bonds. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. A halo-alkenyl group can be any alkenyl group substituted with any number of halogen atoms. “Substituted alkene” can include alkene substituted at 1 or more, e.g., 1, 2, 3, 4, or 5 positions, which substituents are attached at any available atom to produce a stable compound, with substitution as described herein. “Optionally substituted alkene” can include alkene or substituted alkene. Likewise, “alkenylene” can refer to divalent alkene. Examples of alkenylene include without limitation, ethenylene (—CH═CH—) and all stereoisomeric and conformational isomeric forms thereof. “Substituted alkenylene” can refer to divalent substituted alkene. “Optionally substituted alkenylene” can refer to alkenylene or substituted alkenylene.

An “ester” is represented by the formula —OC(O)R, where R can be an alkyl, alkenyl, or group described above.

Alkyne or “alkynyl” refers to a straight, branched chain, or cyclic unsaturated hydrocarbon having the indicated number of carbon atoms and at least one triple bond. The triple bond of an alkyne or alkynyl group can be internal or terminal. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below. An alkyne or alkynyl group can be, for example, a C₂, C₃, C₄, C₅, C₆, C₇, C₈, C₉, C₁₀, C₁₁, C₁₂, C₁₃, C₁₄, CM, CM, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. A halo-alkynyl group can be any alkynyl group substituted with any number of halogen atoms. The term “alkynylene” refers to divalent alkyne. Examples of alkynylene include without limitation, ethynylene, propynylene. “Substituted alkynylene” refers to divalent substituted alkyne.

“PEG” refers to polyethylene glycol. “PEGylated” refers to a compound comprising a moiety, comprising two or more consecutive ethylene glycol moieties. Non-limiting examples of PEG moieties for PEGylation of a compound include, one or more blocks of from 1 to 200 ethylene glycol units, such as —(O—CH₂—CH₂)ₙ—, —(CH₂—CH₂—O)ₙ—, or —(O—CH₂—CH₂)ₙ—OH, where n ranges, for example and without limitation, from 1 to 200 or from 1 to 100, for example from 1 to 5, or 1.

“Aryl,” alone or in combination refers to an aromatic ring system such as phenyl or naphthyl. “Aryl” also can include aromatic ring systems that are optionally fused with a cycloalkyl ring. A “substituted aryl” is an aryl that is independently substituted with one or more substituents attached at any available atom to produce a stable compound, wherein the substituents are as described herein. The substituents can be, for example, hydrocarbyl groups, alkyl groups, alkoxy groups, and halogen atoms. “Optionally substituted aryl” refers to aryl or substituted aryl. An aryloxy group can be, for example, an oxygen atom substituted with any aryl group, such as phenoxy. An arylalkoxy group can be, for example, an oxygen atom substituted with any aralkyl group, such as benzyloxy. “Arylene” denotes divalent aryl, and “substituted arylene” refers to divalent substituted aryl. “Optionally substituted arylene” refers to arylene or substituted arylene. A “polycyclic aryl group” and related terms, such as “polycyclic aromatic group” refers to a group composed of at least two fused aromatic rings. “Heteroaryl” or “hetero-substituted aryl” refers to an aryl group substituted with one or more heteroatoms, such as N, O, P, and/or S. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

“Cycloalkyl” refers to monocyclic, bicyclic, tricyclic, or polycyclic, 3- to 14-membered ring systems, which are either saturated, or partially unsaturated. The cycloalkyl group may be attached via any atom. Cycloalkyl also contemplates fused rings wherein the cycloalkyl is fused to an aryl or hetroaryl ring. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein below. “Cycloalkylene” refers to divalent cycloalkyl. The term “optionally substituted cycloalkylene” refers to cycloalkylene that is substituted with at least 1, 2 or 3 substituents, attached at any available atom to produce a stable compound, wherein the substituents are as described herein.

“Carboxyl” or “carboxylic” refers to group having an indicated number of carbon atoms, where indicated, and terminating in a —C(O)OH group, thus having the structure —R—C(O)OH, where R is an unsubstituted or substituted divalent organic group that can include linear, branched, or cyclic hydrocarbons. Non-limiting examples of these include: C₁-8 carboxylic groups, such as ethanoic, propanoic, 2-methylpropanoic, butanoic, 2,2-dimethylpropanoic, pentanoic, etc. “Amine” or “amino” refers to group having the indicated number of carbon atoms, where indicated, and terminating in a —NH₂ group, thus having the structure —R—NH₂, where R is a unsubstituted or substituted divalent organic group that, e.g. includes linear, branched, or cyclic hydrocarbons, and optionally comprises one or more heteroatoms. The term “alkylamino” refers to a radical of the formula —NHR^{x} or —NR^{x}R^{x} where each R^{x} is, independently, an alkyl radical as defined above.

Terms combining the foregoing refer to any suitable combination of the foregoing, such as arylalkenyl, arylalkynyl, heteroarylalkyl, heteroarylalkenyl, heteroarylalkynyl, heterocyclylalkyl, heterocyclylalkenyl, heterocyclylalkynyl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkyl heterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl. As an example, “arylalkylene” refers to a divalent alkylene wherein one or more hydrogen atoms in an alkylene group is replaced by an aryl group, such as a (C₃-C₈)aryl group. Examples of (C₃-C₈)aryl-(C₁-C₆)alkylene groups include without limitation 1-phenylbutylene, phenyl-2-butylene, l-phenyl-2-methylpropylene, phenylmethylene, phenylpropylene, and naphthylethylene. The term “(C₃-C₈)cycloalkyl-(C₁-C₆)alkylene” refers to a divalent alkylene wherein one or more hydrogen atoms in the C₁-C₆ alkylene group is replaced by a (C₃-C₈)cycloalkyl group. Examples of (C₃-C₈)cycloalkyl-(C₁-C₆)alkylene groups include without limitation 1-cycloproylbutylene, cycloproyl-2-butylene, cyclopentyl-1-phenyl-2-methylpropylene, cyclobutylmethylene and cyclohexyl propylene.

A fatty acid is an aliphatic monocarboxylic acid, comprising a carboxyl group linked to an aliphatic hydrocarbyl group which may be saturated or unsaturated. A hydrocarbyl or hydrocarbon group refers to a group of carbon and hydrogen atoms, such as alkyl, alkenyl, or aryl groups. By “aliphatic”, it is meant acyclic or cyclic, saturated or unsaturated hydrocarbon compounds, excluding aromatic compounds. The aliphatic group of fatty acids is typically a linear chain of carbons, but fatty acids and substituted fatty acids as a class include linear, branched, and/or cyclic carbon chains. As used herein, fatty acids include both natural and synthetic aliphatic carboxylic acids. Fatty acids can have an aliphatic chain of from four to 24 carbon atoms (for example, as used herein, “a (C₄-C₂₄) fatty acid”). Hydrogen atoms of a compound, such as a fatty acid may be substituted with a group or moiety (hereinafter referred to as a “substituent”), to produce a substituted fatty acid. Fatty acids and substituted fatty acids may be referred to as “optionally substituted fatty acids”) Non-limiting examples of substituents include nitro groups (—NO₂, with the dash indicating connection to another chemical moiety, such as an aliphatic hydrocarbyl chain of a fatty acid), or hydroxyl groups (—OH), among others. Fatty acids, and fatty acid groups, may be referred to by the number of carbon atoms and the number of double bonds, e.g., C10:0, referring to a fatty acid or fatty acid group having 10 carbon atoms and zero double bonds. Likewise, C18:1 refers to a fatty acid with an 18-carbon chain having one double bond, such as oleic acid.

Unsaturated fatty acids and substituted unsaturated fatty acids (collectively “optionally substituted unsaturated fatty acids”) comprise one or more carbon-carbon double bonds, or an alkenyl group (also, vinyl group) in their aliphatic chains. The individual carbon atoms of the alkenyl group are referred to herein as alkenyl carbons. Unless specified, any carbon-carbon double bond in the alkyl chain of the described optionally substituted unsaturated fatty acids independently may be E (trans) or Z (cis) geometric isomers, or mixtures thereof.

The structure of 10-nitrolinoleic acid ((9E,12Z)-10-nitrooctadeca-9,12-dienoic acid), having double bonds at the 9 and 12 carbon, characteristic of linoleic acid, is exemplified in the following structure, depicting typical fatty acid numbering scheme:

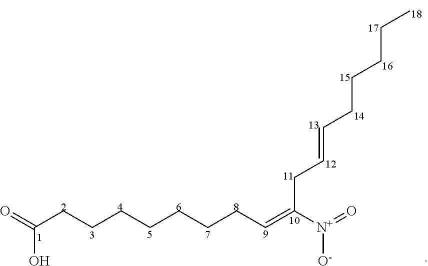 [SMILES: CCCCC/C=C/C/C(=C\CCCCCCCC(=O)O)[N+](=O)[O-]]

The structure of 9-nitrooleic acid ((E)-9-nitrooctadec-9-enoic acid), with a double bond at the 9 carbon characteristic of oleic acid, is depicted in the following structure:

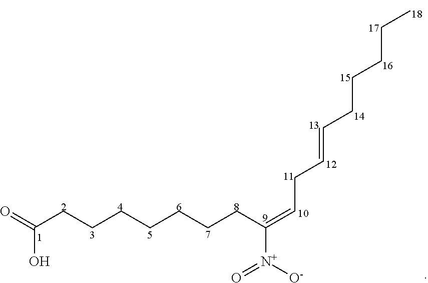 [SMILES: CCCCC/C=C/C/C=C(\CCCCCCCC(=O)O)[N+](=O)[O-]]

Compounds described herein, including fatty acids and substituted fatty acids such as nitro fatty acids can exist in various isomeric forms, including configurational, geometric, and conformational isomers, as well as existing in various tautomeric forms, such as those that differ in the point of attachment of a hydrogen atom. The term “isomer” is intended to encompass all isomeric forms of a compound of this invention, including tautomeric forms of the compound.

Certain compounds described here may have asymmetric centers and therefore exist in different enantiomeric and diastereomeric forms. A compound can be in the form of an optical isomer or a diastereomer. Accordingly, compounds described herein include their optical isomers, diastereoisomers and mixtures thereof, including a racemic mixture unless otherwise specified. Optical isomers of the compounds of the invention can be obtained by known techniques such as asymmetric synthesis, chiral chromatography, simulated moving bed technology or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

A nitro fatty acid (e.g., a nitroalkene fatty acid) is a substituted fatty acid substituted with one or more nitro groups (—NO₂). The nitro fatty acid may be saturated. The nitro fatty acid may be unsaturated. The nitro group may be attached to an alkenyl carbon of the nitro fatty acid, such as the 9 or 10 carbon of oleic acid, or the 9, 10, 12, or 13 carbon of linoleic acid. The nitro-fatty acid may have a carbon-carbon double bond with an NO₂ substituent attached to a first carbon adjacent to (that is, alpha to) a first carbon of the carbon-carbon double bond and an ONO₂ substituent attached to a second carbon alpha to the second carbon of the carbon-carbon double bond, that is:

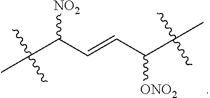

See also, Formula III, below. For use in the compositions and methods described herein, the nitro-fatty acid may be anti-inflammatory, or have other therapeutic benefits. U.S. Pat. No. 8,735,449, along with International Patent Publication No. WO 2009017802 and U.S. Pat. Nos. 9,186,408, 9,700,534, 10,258,589, and 10,576,051, the disclosure of each of which is incorporated herein by reference in its entirety, describes use of nitro fatty acids, such as 9- or 10-nitro octadecenoic acid (examples of “nitro oleic acids”) and various regioisomers (9-, 10-, 12- and 13-nitro) of nitro linoleic acid, and pharmaceutically acceptable salts, metabolites, and prodrugs thereof, as having anti-inflammatory effect. Prodrugs of fatty acids include esters, such as glyceryl esters thereof (e.g., mono-, di-, and tri-glycerides including nitro-fatty acid groups).

WO 2010/014889 A2, the disclosure of which is incorporated by reference in its entirety, discloses nitro fatty acid mimetics that activate PPAR-γ (peroxisome proliferator activated receptor gamma). Nitro-linoleic acid and nitro-oleic acid, illustrative of the mimetic category, are described as nanomolar activators of the PPAR-γ receptor. For example, it was shown that 10-nitro linoleic acid and 12-nitro linoleic acid are potent, selective activators of the PPAR-γ receptor.

A non-limiting list of useful nitro-fatty acids for the compositions and methods described herein includes: 9-nitrooleic acid, 10-nitrooleic acid, 9-nitrolinoleic acid, 10-nitrolinoleic acid, 12-nitrolinoleic acid, 13-nitrolinoleic acid, 9-Nitrononanoic acid, 9-nitro-9-trans-octadecenoic acid, 10-nitro-9-trans-octadecenoic acid, or a salt or ester thereof, e.g., an allyl ester thereof or an amphiphilic ester thereof, such as a phospholipid, such as, for example, phospholipids comprising a nitro-fatty acid moiety ester-linked to the phospholipid, or other amphipathic compounds comprising an ester-linked nitro-fatty acid moiety, e.g., as described herein. Phospholipids may comprise a glycerol moiety (phosphoglycerides) to which fatty acid groups or moieties are ester-linked, in which case at least one nitro-fatty acid moiety, such as, without limitation, 9-nitrooleic acid, 10-nitrooleic acid, 9-nitrolinoleic acid, 10-nitrolinoleic acid, 12-nitrolinoleic acid, 13-nitrolinoleic acid, 9-Nitrononanoic acid, 9-nitro-9-trans-octadecenoic acid, or 10-nitro-9-trans-octadecenoic acid, is ester-linked to the phospholipid. For example a nitro-fatty acid may be ester-linked to a phospholipid at the sn2 position of the glycerol moiety of the phospholipid. Many nitro-fatty acids are available commercially, e.g., from Cayman Chemical of Ann Arbor, Mich., or can be made by known methods.

Additional non-limiting examples of nitro-fatty acids include Formulas I-IV, below. The nitro-fatty acid may have the structure of Formula I:

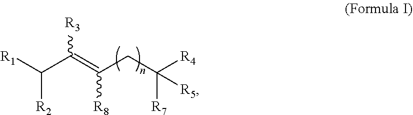

wherein R₁ is hydrogen, C₁-C₂₄ alkyl or C₁-C₂₄ alkenyl; R₂, R₃, R₇, and R₈ are each independently, hydrogen, oxygen, C₁-C₂₄ alkyl or NO₂; R₄ is a terminal COOR₆ group, wherein R₆ is hydrogen, C₁-C₂₄ alkyl, or a pharmaceutically acceptable counterion; R₅ is hydrogen, C₁-C₂₄ alkyl, or R₄ and R₅ collectively form ═C(R₉)(R₁₀), wherein R₉ comprises C₁-C₂₄ alkyl or C₁-C₂₄ alkenyl, or wherein R₉ is a terminal COOR₆ group, and R₁₀ is hydrogen or NO₂; n is from 1 to 24; and wherein at least one of R₂, R₃, R₇, R₈, and R₁₀, when present, is an NO₂ group. In certain embodiments, R₁ is C₁-C₂₄ alkyl, more particularly C₃-C₂₀ alkyl. In certain embodiments, R₂ is hydrogen. In certain embodiments, one of R₃ or R₈ is NO₂ and the other of R₃ or R₈ is hydrogen. In certain embodiments, n is from 3 to 20. In certain embodiments, R₄ is —COOH. In certain embodiments, R₅ is hydrogen. In certain embodiments, R₇ is hydrogen. In certain embodiments, R₄ is —COOH; R₅ is methyl; and R₇ is methyl. Wavy lines in any chemical structure provided herein indicates any stereoisomers, e.g., E and Z isomers, including either or both stereoisomers in any ratio.

The nitro-fatty acid may have the structure of Formula II:

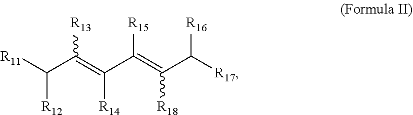

wherein R₁₁ is hydrogen, C₁-C₂₄ alkyl, or C₁-C₂₄ alkenyl; R₁₂, R₁₄, R₁₅ and R₁₆ are hydrogen, C₁-C₂₄ alkyl, or C₁-C₂₄ alkenyl; R₁₃ and R₁₈ are each independently, hydrogen, oxygen, C₁-C₂₄ alkyl, or NO₂; R₁₇ is a terminal COOR₁₉ group, wherein R₁₉ is hydrogen, C₁-C₂₄ alkyl, or a pharmaceutically acceptable counterion, wherein at least one of R₁₃, R₁₄, R₁₅, and R₁₈ is NO₂.

The nitro-fatty acid may have the structure of Formula III:

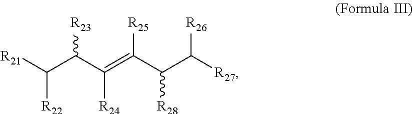

wherein R₂₁ is hydrogen, C₁-C₂₄ alkyl, or C₁-C₂₄ alkenyl; R₂₂, R₂₄, R₂₅′ and R₂₆ are hydrogen; one of R₂₃ and R₂₈ is NO₂, and the other of R₂₃ and R₂₈ is ONO₂; and R₂₇ is a terminal COOR₂₉ group, wherein R₂₉ is hydrogen, C₁-C₂₄ alkyl, or a pharmaceutically acceptable counterion.

In certain embodiments, the nitro-fatty acid is 10-nitro-octadec-9-enoic acid (NO₂-OA). In certain embodiments, the nitro-fatty acid is 9-nitro-octadec-9-enoic acid (NO₂-OA). In certain embodiments, the nitro-fatty acid is 8-nitro-nonadec-9-enoic acid. In certain embodiments, the nitro-fatty acid is 7-NO₂-nonadec-7-enoic acid. In certain embodiments, the nitro-fatty acid is 5-NO₂-eicos-5-enoic acid or 6-NO₂-eicos-5-enoic acid. In certain embodiments, the nitro-fatty acid is substantially pure. In this aspect, the stereochemistry about the carbon-carbon double bond may be substantially cis (or Z) or substantially trans (or E). In certain embodiments, the nitro-fatty acid is 9-nitrooctadeca-9,11-dienoic acid In certain embodiments, the nitro-fatty acid is 12-nitrooctadeca-9,11-dienoic acid. In certain embodiments, the nitro-fatty acid is 9-nitro-12-(nitrooxy)octadec-10-enoic acid. In certain embodiments, the nitro-fatty acid is 12-nitro-9-(nitrooxy)octadec-10-enoic acid.

Lipids as a group includes glycerides and phospholipids. A “glyceride” is an ester of glycerol (propane 1,2,3-triol) with a fatty acid or a substituted fatty acid. Phospholipids are lipids containing phosphoric acid as mono- or di-esters, such as phosphatidic acids and phosphoglycerides. Phosphoglycerides are di-esters of glycerol, which are glycerol derivatives in which one hydroxyl group of the glycerol is phosphodiester-linked to a group, such as a functional group, such as, for example and without limitation, a 2-amino ethanol or a choline (e.g., —O—CH₂—CH₂—N*(CH₃)₃) groups. A phosphatidylcholine is a phosphoglyceride with a choline linked to the glycerol moiety by a phosphodiester linkage. A glycerol-phosphoethanolamine is a phosphoglyceride with an 2-amino ethane group (e.g., —CH₂—CH₂—NH₃) linked to the glycerol moiety by a phosphodiester linkage. As described above, one or more nitro-fatty acids may be incorporated into the phospholipid or phosphoglyceride, or generally into any amphipathic lipid comprising an ester-linked nitro-fatty acid moiety. Amphipathic refers to a molecule or compound having both hydrophobic and hydrophilic parts, e.g., under physiological conditions.

The microbubbles described herein can comprise a lipid-based shell comprising a circumferential region that defines (e.g., encapsulates and stabilizes) a core of the microbubble, a gas within the core of the microbubble, and a nitro-fatty acid incorporated in the lipidic microbubble shell. Alternatively, where the NFA is administered separately from the microbubble, the microbubble can comprise a lipid-based shell comprising a circumferential region that defines a core of the microbubble, and a gas within the core of the microbubble. The gas may comprise air, oxygen, nitrogen, argon, or a perfluorocarbon, such as perfluorobutane, perfluoropropane, octafluoropropane, perfluorohexane, perfluorodiglyme, and/or perfluorotriglyme. The lipid-based shell can comprise any stable combination of lipids, such as phospholipids, PEGylated phospholipids, phosphatidylcholine, a PEGylated fatty acid, and where included, a nitro-fatty acid, or an ester, optionally an amphiphilic ester or an allyl ester, comprising a nitro fatty acid. Lipid molar ratio may affect back-scattered power, most likely due to different shell viscosity. Longer hydrophobic chains in the phospholipid layer may lead to increased acoustic emissions and drug delivery, while longer acyl chains may be used to increase lipid-shelled particle stability at in vivo. PEG also provides a steric brush that minimizes lipid shell interactions with blood components.

The lipid-based shell can comprise a phospholipid, a phosphotidylcholine, and/or a glycerol-phosphoethanolamine lipid that is optionally PEGylated. The lipid-based shell may comprise a PEGylated fatty acid. In one example, the lipid-based shell comprises a phospholipid, a phosphotidylcholine, a glycerol-phosphoethanolamine lipid, and a PEGylated fatty acid. The lipid-based shell may comprise: 1,2-distearoyl-sn-glycero-3-phosphocholine, e.g.,

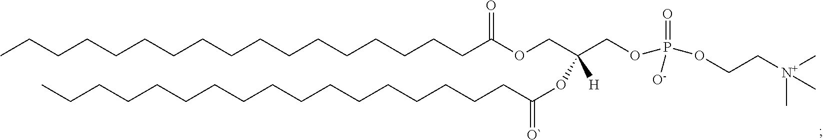

1,2-Distearoyl-sn-Glycero-3-Phosphoethanolamine-N-[Methoxy(Polyethylene glycol)-2000], e.g.,

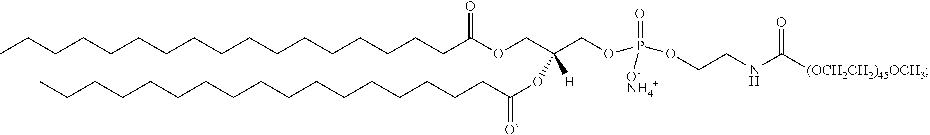

and PEG-stearate, e.g. polyoxyethylene (40) stearate:

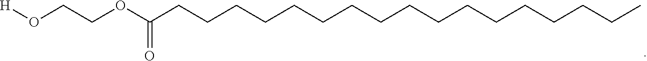 [SMILES: CCCCCCCCCCCCCCCCCC(=O)OCCO]

The lipid-based shell may comprise: 1,2-distearoyl-sn-glycero-3-phosphocholine; 1,2-Distearoyl-sn-Glycero-3-Phosphoethanolamine-N-[Methoxy(Polyethylene glycol)-2000]; and PEG-stearate, e.g. polyoxyethylene (40) stearate in a molar ratio of 0.026/0.003/0.022 moles (or 2/1/1 by weight ratio), e.g. with at least 0.015 moles of NFAB (12.5% by weight ratio). For example, 15 mg of 1,2-distearoyl-sn-glycero-3-phosphocholine, 10 mg of 1,2-Distearoyl-sn-Glycero-3-Phosphoethanolamine-N-[Methoxy(Polyethylene glycol)-2000] and 10 mg of PEG-stearate with 5 mg of NO₂-OA. Where nitro-fatty acid or ester thereof is associated with the lipid-based shell, therapeutic, e.g., anti-inflammatory, amounts of the nitro-fatty acid are included, such as, for example a ratio as compared to other lipids present in the lipid shell ranging from 10 to 25% by mass or from 20 to 40% by moles, from 7.5 to 17.5% by mass or from 15 to 30% by moles, from 10 to 15% by mass or from 18 to 25% by moles, for example about 12.5% by mass and about 21.9% by moles or 12.5% by mass and 21.9% by moles.

Where nitro-fatty acid is associated with the lipid-based shell, therapeutic, e.g., anti-inflammatory, amounts of the nitro-fatty acid are included, such as a molar ratio as compared to other lipids present in a lipid shell comprising: 1,2-distearoyl-sn-glycero-3-phosphocholine; 1,2-Distearoyl-sn-Glycero-3-Phosphoethanolamine-N-[Methoxy(Polyethylene glycol)-2000]; and PEG-stearate, e.g. polyoxyethylene (40) stearate, the molar ratio of 1,2-distearoyl-sn-glycero-3-phosphocholine:1,2-Distearoyl-sn-Glycero-3-Phosphoethanolamine-N-[Methoxy(Polyethylene glycol)-2000]: PEG-stearate, e.g. polyoxyethylene (40) stearate:nitro-fatty acid may range from 10 to 25% by mass or from 20 to 40% by moles, from 7.5 to 17.5% by mass or from 15 to 30% by moles, from 10 to 15% by mass or from 18 to 25% by moles, for example about 12.5% by mass and about 21.9% by moles or 12.5% by mass and 21.9% by moles.

Where nitro-oleic acid or nitro-linoleic acid is associated with the lipid-based shell, therapeutic, e.g., anti-inflammatory, amounts of the nitro-fatty acid are included, such as a molar ratio as compared to other lipids present in a lipid shell comprising: 1,2-distearoyl-sn-glycero-3-phosphocholine; 1,2-Distearoyl-sn-Glycero-3-Phosphoethanolamine-N-[Methoxy(Polyethylene glycol)-2000]; and PEG-stearate, e.g. polyoxyethylene (40) stearate, the molar ratio of 1,2-distearoyl-sn-glycero-3-phosphocholine:1,2-Distearoyl-sn-Glycero-3-Phosphoethanolamine-N-[Methoxy(Polyethylene glycol)-2000]:PEG-stearate, e.g. polyoxyethylene (40) stearate:nitro-fatty acid may range from 10 to 25% by mass or from 20 to 40% by moles, from 7.5 to 17.5% by mass or from 15 to 30% by moles, from 10 to 15% by mass or from 18 to 25% by moles, for example about 12.5% by mass and about 21.9% by moles or 12.5% by mass and 21.9% by moles.

Perfluorocarbons are clear, odorless liquids composed primarily of covalently bonded carbon and fluorine atoms, including ethers thereof. A perfluorocarbon having one or more ether linkages is termed a “perfluoroether”, and can be cyclic (perfluorocycloether). Perfluorocarbons include single compounds and mixtures of two or more perfluorocarbon compounds. Perfluorocarbons are broadly-known and many are commercially available, including those that have been studied for medical or pharmacological purposes, including breathing, respiratory therapies, imaging, and drug delivery. Perfluorocarbons as a class include substituted perfluorocarbons in which one or more heteroatoms are added, such as, for example and without limitation, O in the case of ethers, and Br, e.g., in the case of Perflubron (perfluorooctylbromide). Perfluorocarbons may be linear, branched, cyclic, or combinations thereof and are saturated, and in various aspects include from six to twelve carbon atoms. Perfluorocarbons may be biocompatible, non-toxic, and/or pharmaceutically-acceptable.

Provided herein are microbubbles comprising: a lipid-based shell that defines, e.g. encapsulates and stabilizes, a core of the microbubble, a gas within the core of the microbubble, and a nitro-fatty acid associated with the microbubble. By associated, it is meant incorporated into, admixed into absorbed into, adsorbed onto, or otherwise linked to or mixed into a component of the microbubble. The microbubbles may be used for localized delivery of the nitro fatty acid in a patient, for example in the vascular system of a patient. The use of lipid-based microbubbles in ultrasound imaging and therapy is described in the literature, and while the microbubble composition may be optimized for any specific nitro-fatty acid, the composition of the microbubbles described herein are intended to be illustrative.

U.S. Pat. No. 10,413,278, the disclosure of which is incorporated herein by reference, provides a method of treating a thrombotic condition by performing ultrasound contrast assisted therapy using microbubble-based contrast agents that do not comprise nitro-fatty acids. In a similar manner, the microbubbles described herein, containing the nitro-fatty acid, may be delivered to a target area of a patient, for example using a catheter. The target area may comprise a thrombus or MVO which is in a vessel or a microvessel of the patient. The microbubble may be disrupted by delivering an ultrasonic pulse or a sequence of ultrasonic pulses (e.g. tones or tone bursts) to the target area from a location external to the vessel or microvessel or from within the vessel or microvessel.

The ultrasound device produces oscillations of the microbubble, e.g., against the thrombus or MVO. The pulse sequence may be any useful pulse sequence for disrupting the microbubbles as described herein. For example ultrasound may be applied continually or as a single burst, or in multiple tone burst patterns comprising two or more tone bursts that may be the same or different. A tone burst may be, for example and without limitation, from 1 microsecond (μs) to 100 seconds (s). Tone bursts may be applied repeatedly at a pulse repetition frequency ranging from, for example and without limitation, 0.001 Hz to 100 Hz, or 0.01 Hz to 20 Hz. The pressure amplitude of the tone bursts may be greater than 0.1 MPa, greater than 0.25 MPa, greater than 0.5 MPa, or greater than 1.0 MPa for the duration of each of the ultrasound tone bursts. The ultrasound frequency may range between 0.25 MHz and 10 MHz, preferably between 0.5 MHz to 5 MHz. The pulse sequence may have a time averaged ultrasound intensity that is less than 1.0 W/cm², less than 0.75 W/cm², or less than 0.5 W/cm². Tone bursts may be applied in sweeps of ultrasound frequency or other parameters such as pulse repetition frequency, pulse duration, pressure amplitude, or time-averaged ultrasound intensity, sweeping from high to low, or low to high values. Other patterns of tone bursts may be employed. The choice of pattern of ultrasound tone bursts, including use of continuous wave over a limited amount of time, may be optimized based on, for example and without limitation, the composition of the microbubble and the tissue to which the microbubble is locally-delivered.

Ultrasound devices useful for disrupting the microbubbles described herein are broadly-available, and include devices for administering ultrasound externally to the patient, or internally to the patient. If internal, the ultrasound device may be catheter-delivered ultrasound, e.g., ICE (intra cardiac ultrasound) or IVUS (intravascular ultrasound, see, e.g., Gentry K L, et al., Catheter ultrasound phased-array transducers for thermal ablation: a feasibility study. Ultrason Imaging. 2005 April; 27(2):89-100. doi: 10.1177/016173460502700203. PMID: 16231838 and Xiaohua Jian, et al., “A High Frequency Geometric Focusing Transducer Based on 1-3 Piezocomposite for Intravascular Ultrasound Imaging”, BioMed Research International, vol. 2017, Article ID 9327270, 8 pages, 2017).

In the embodiments disclosed herein, the specific attributes of the pulse waveform can vary based on the frequency desired for certain tissue types and organ size, the type of microbubble used, and theultrasoundpressure amplitude. For example, typically, a tone burst of greater than 5 acoustic cycles may be used. For example, typically, a tone burst ranging from 10-5,000 acoustic cycles is used (with a range of 1,000-2,000 acoustic cycles being preferred for some embodiments) with a pulse repetition frequency (PRF) that allows replenishment of the microbubble into the region of interest (ROI). In some embodiments, a PRF range of 0.01-20 Hz can be used, with a range of 0.2-1 Hz being preferred in certain embodiments. An ultrasound frequency of 0.25 MHz-10 MHz can be used in some embodiments, with a frequency range of 0.5-5 MHz being preferred for certain embodiments, as those frequencies are close to the resonance frequency of some of the microbubbles present in contrast agents described herein. In addition, pressure amplitude of greater than 0.3 MPa can be used. In addition, a pressure amplitude of 0.5-1.9 MPa (and, more preferably 1.5 MPa in some embodiments) can be used.

In some embodiments, the methods and systems described herein can provide improved therapy by reducing systemic exposure and delivery of large amounts of nitro-fatty acids in a patient. In addition, the relatively low PRF reduces the total amount of ultrasound energy and therefore reduces at least some potential bioeffects, such as heating. For example, when a 2,000 acoustic cycle tone burst of 1.5 MPa acoustic pressure is used at a PRF of 0.5 Hz, the duty cycle is 0.1%, and the time averaged ultrasound intensity is only 0.075 W/cm².

In some embodiments, a device is provided that includes an ultrasound generator and ultrasound transducer that can produce ultrasound waveforms. The ultrasound generator can be configured such that the ultrasound pulse configuration can be changed online according to specific microbubble properties and blood flow conditions. This can permit the total ultrasound energy to be minimized while still harnessing most of the bubble activity for therapeutic purposes. The ultrasound generator can be a self-contained unit or an attachment or an add-on of another system such as an ultrasound imaging system. The ultrasound transducer can be a single element transducer with or without focusing. For example, the transducer can be a spherically-focused or cylindrically-focused transducer depending on the type of tissue/or organ to be treated. The transducer can also be formed by an array of elements to allow dynamic focusing of the treatment region of interest (ROI).

The transducer can also be constructed to allow imaging of microbubble activity or for cavitation detection. For this mode of application, the transducer can be broadband or capable of dual frequency applications such that better image resolution is achieved with high frequency. The ultrasound transducer can also be designed such that imaging can be performed with third party imaging systems.

The therapy described herein may be used to treat conditions associated with an inflammation response, or inflammatory conditions, and the delivery method, and ultrasound devices may be selected to optimize local delivery of the microbubbles and application of the ultrasound. The inflammatory condition may be associated with ischemia or ischemia-reperfusion, such as in the case of myocardial infarct, thrombosis or microvascular obstructions following thrombolysis, stroke, stenting, stenosis or re-stenosis, placement of an embolism coil, heart surgery such as, without limitation, valve replacement, tumor resection or ablation, treatment of local infection or abscess, traumatic injury, or surgical procedure.

The microbubble compositions and methods of use of those compositions may be used in a thrombolysis procedure, or in a procedure to treat microvascular obstruction. The treatment may be performed, for example and without limitation, in the heart, vasculature, peripheral vasculature, the lungs, or in the brain.

The microbubbles described herein also may be used to treat fibrosis in a patient. The fibrosis may be pulmonary fibrosis, liver fibrosis, scarring, or any other fibrotic lesion in the patient. Nitro-fatty acids have demonstrable effect in reducing inflammation and even in reversal of fibrosis, see Example 4, below. Ablation of thrombi and fibrotic lesions may be performed using ultrasound. However, use of ultrasound ablation techniques with or without microbubbles, can result in fibrosis (See, e.g., Nazer B, Microbubble-Facilitated Ultrasound Catheter Ablation Causes Microvascular Damage and Fibrosis. Ultrasound Med Biol. 2021 January; 47(1):131-138. doi: 10.1016/j.ultrasmedbio.2020.09.007. Epub 2020 Oct. 20. PMID: 33092899). Inflammation is associated with development of fibrotic lesions. As such, ablation of fibrotic lesions, or treatment of fibrotic lesions with NFAs and microbubbles according to any method or using any composition as described herein, in conjunction with ultrasound effective to disrupt the microbubbles at the site of the fibrotic lesion, thereby releasing the NFAs at the site of the fibrotic lesion is expected to reduce fibrosis. The NFAs or small-molecule nitroalkenes may be associated with the microbubble, or may be co-administered as a free fatty acid or free nitroalkene compound along with a microbubble that is not associated with the fatty acid or nitroalkene compound.

The microbubbles described herein, including or without NFAs or esters thereof, may be co-administered with, may include, or may be attached to least one therapeutically active agent and optionally to one or more additional therapeutically active agents, targeting agents, and/or visualization agents. Modification of microbubbles is described in United States Patent Application Publication No. 20200338172, the disclosure of which is incorporated herein by reference for its disclosure of microbubbles, modified microbubbles, and methods of preparing modified microbubbles. The therapeutically active agent can be a thrombolytic agent capable of reacting with a thrombus to promote disruption, degradation, and/or destruction of the thrombus (e.g., to reduce the size of the thrombus such that blood perfusion is restored). The thrombolytic agent can be a fibrinolytic agent, such as tissue plasminogen activator (“tPA”), a recombinant tissue plasminogen activator (e.g., Alteplase, Reteplase, Tenecteplase, Desmoteplase, or the like), a streptokinase activator (e.g., Anistreplase), or any combination thereof. The thrombolytic agent may be tPA. Exemplary additional therapeutically active agents that can be coupled to the microbubble include, but are not limited to, anticancer agents, antiplatelet drugs (e.g., aspirin, an adenosine diphosphate receptor inhibitor, a phosphodiesterase inhibitor, a protease-activated receptor-1 antagonist, a glycoprotein IIB/IIIA inhibitor, an adenosine reuptake inhibitor, or a thromboxane inhibitor), a protein (e.g., an antibody), a gene, a vector, or combinations thereof. Exemplary targeting agents can include, but are not limited to, p-selectin and other compounds that can target areas within a vascular or microvascular structure, which can, in some embodiments, include a glycoprotein IIB/IIIA inhibitor and/or an antibody. Exemplary visualization agents can include, but are not limited to, contrast agents, dyes, fluorophores, quantum dots, or other visually detectable agents.

The microbubble may be attached to least one therapeutically active agent using specific binding pair chemistry, such as biotin/streptavidin (or avidin, or a deglycosylated version thereof) chemistry. The microbubble may be attached to at least one therapeutically active agent via one or more covalent bonds formed between covalent binding partners, such as an organic functional group of a lipid of the microbubble and an organic functional group of the therapeutically active agent. In some examples, a linker can be used as spacer positioned between (and bound to) the microbubble and the therapeutically active agent. Exemplary covalent binding partners that can be used include thiol groups which can covalently bind with sulfur-reactive groups (e.g., another thiol group, a disulfide group, a carbon-carbon double bond, or a carbon-halide bond, such as —C(R₂)I, —C(R₂)Br, —C(R₂)F, —C(R₂)Cl, wherein each R₂ independently is hydrogen, aliphatic, heteroaliphatic, haloaliphatic, haloheteroaliphatic, aromatic, or a combination thereof) and/or amine groups that can covalently bind with amine-reactive groups (e.g., isothiocyanates, isocyanates, sulfonyl chlorides, aldehydes, acyl azides, anhydrides, carbonates, NHS esters, imidoesters, epoxides, fluorophenyl esters or benzene, carboxylic acids, carboxylic acids modified with a carbodiimide, and the like).

Exemplary covalent binding partners can include a maleimide group present on a microbubble (as one partner) and a thiol group present on a therapeutically active agent (as the other partner); and/or a carboxylic acid group present on a microbubble (as one partner) that can be coupled with a carbodiimide (e.g., 1-Ethyl-3-(3-dimethylaminopropyl)carbodiimide, or “EDC”; or dicyclohexylcarbodiimide or “DCC”) and a primary amine group present on a therapeutically active agent (as the other partner). The microbubble can be coupled to a liposome that comprises a therapeutically active agent encapsulated therein. The liposome can be attached to the microbubble, e.g., using coupling techniques disclosed herein.

If a linker is included, it can be a linker comprising an aliphatic group, a heteroaliphatic group, an aromatic group, a haloaliphatic group, an organic functional group, or any combinations thereof. The linker can be selected to provide a particular distance between the microbubble and the therapeutically active agent, such as by increasing or decreasing the length of the linker. The linker can be selected to impart a particular solubility to the functionalized microbubble, such as by selecting a substantially hydrophilic linker or a substantially hydrophobic linker, or even a combination thereof. For example, the linker can be 4-ethyl-N-(4-pentanamidobutyl)cyclohexane-1-carboxamide or a polyethylene glycol (or “PEG”) group (e.g., PEG4). Similar chemical attachments and linkers can be used to functionalize the microbubble with one or more of an additional therapeutically active agent, a visualization agent, a targeting agent, and any combinations thereof.

In particular examples, the therapeutically active agent is a thrombolytic agent, such as tPA, and the thrombolytic agent is covalently attached directly or indirectly (such as through a linker group) to a biotin molecule to provide a biotin-functionalized therapeutically active agent. In such embodiments, the microbubble comprises a lipid-based shell that also is functionalized with one or more biotin molecules. The lipid-based shell of the microbubble can be further modified to comprise one or more streptavidin molecules, and/or avidin molecules (or a deglycosylated version thereof) that specifically bind to the biotin molecules attached to the microbubble.

In examples, the therapeutically active agent may be a thrombolytic agent, such as tPA, and the thrombolytic agent is covalently attached directly or indirectly (such as through a linker) to one or more lipids of the microbubble via a thiol moiety of the thrombolytic agent. In such embodiments, the microbubble comprises one or more lipids that are attached directly or indirectly (such as through a linker) to one or more maleimide groups. The thiol group of the thrombolytic agent can covalently bind to the carbon-carbon double bond of the maleimide group, thereby forming a covalent linkage to the lipid-bases shell of the microbubble.

In examples, the therapeutically active agent is a thrombolytic agent, such as tPA, and the thrombolytic agent is covalently attached directly or indirectly (such as through a linker) to one or more lipids of the microbubble via an amine moiety of the thrombolytic agent. In such embodiments, the microbubble comprises one or more lipids that have a carboxylic acid group (or that comprise one or more linkers having carboxylic acid groups). Using amide bond-forming reagents (e.g., EDC or DCC in combination with n-hydroxysuccinimide or “NHS”), the amine group of the thrombolytic agent can form an amide bond with the carboxylic acid group of the microbubble.

In examples, at least one lipid of the lipid-based shell is coupled to the therapeutically active agent. In examples, a plurality of lipids can be coupled to a plurality of therapeutically active agents (which can be the same or different from one another), such that each lipid is coupled to one therapeutically active agent. In some embodiments, the concentration of the therapeutically active agent that is delivered to a subject or sample is controlled by controlling the lipid:therapeutically active agent ratio of the functionalized microbubble. The functionalized microbubble may have a volumetric ratio of therapeutically active agent:microbubble ranging from 1:1 to 6:1, such as 1:1, 3:1, or 6:1. In examples where a thrombolytic agent is employed as a therapeutically active agent, the functionalized microbubble can be made with a lipid-to-thrombolytic agent ratio that provides a local tissue concentration ranging from greater than zero to 3 μg/mL of the thrombolytic agent upon administration to the subject or sample. The lipid-to-thrombolytic agent ratio can be selected to provide a local tissue concentration ranging from 0.1 μg/mL to 2.5 μg/mL, or 0.1 μg/mL to 2 μg/mL, or 0.1 μg/mL to 1.5 μg/mL, or 0.1 μg/mL to 1 μg/mL, or 0.1 μg/mL to 0.5 μg/mL of the thrombolytic agent. The functionalized microbubble may comprise from 1×10⁻⁷ μg to 7×10⁻⁷ μg of the thrombolytic agent (e.g., tPA) per microbubble, such as from 1.2×10⁻⁷ μg to 6.5×10⁻⁷ μg of tPA per microbubble, or from 1.5×10⁻⁷ μg to 6×10⁻⁷ μg of tPA per microbubble. The amount of tPA loaded onto the functionalized microbubble is selected to provide a local tissue concentration (upon administration) that is 1% to 50% less than a typical systemic dose of tPA (which is generally recognized in the art to result in a plasma concentration of 3 μg/mL), such as 1% to 45%, or 1% to 40%, or 1% to 30%, or 1% to 20%, or 1% to 10% (e.g., 1%, 2%, 3%, 4%, 5%, 6%, 7%, 8%, 9%, or 10%) less than a systemic dose. Even at these lower dosages of tPA, the functionalized microbubble is able to promote thrombolysis of a thrombus as effectively, or even more effectively, as the systemic dose of tPA when used in method embodiments disclosed herein.

Compositions comprising the microbubbles, e.g. the functionalized microbubbles can comprise one or more functionalized microbubble embodiments (which can be different, such as including different therapeutically active agents), and a pharmaceutically acceptable excipient, one or more additional therapeutically active agents, one or more visualization agents, one or more non-functionalized microbubbles, and any combinations thereof. Proper formulation of the pharmaceutical composition is dependent upon the route of administration chosen. The composition embodiments disclosed herein may be manufactured in any manner known in the art with the benefit of the present disclosure, such as mixing, dissolving, granulating, dragee-making, levigating, emulsifying, encapsulating, entrapping, or compression processes. Exemplary additional therapeutics can include, but are not limited to, anticancer agents, such as, without limitation, antiangiogenic and antiproliferative agents.

Chronic inflammation has long been a relevant factor in tumor progression. Tumor microenvironment is an important factor in the neoplastic process, and as such anti-inflammatory therapeutics are relevant to the treatment of cancers (see, e.g., Coussens L M, Werb Z. Inflammation and cancer. Nature. 2002; 420(6917):860-867. doi:10.1038/nature01322). The treatment methods described herein, comprising local administration of nitro-fatty acids either incorporated in microbubbles or administered with microbubbles, to the site of a tumor (benign or malignant) or precancerous lesion, followed by ultrasound disruption of the locally-administered microbubbles at the site of the tumor or precancerous lesion, is expected to treat the tumor or precancerous lesion. The microbubble treatment may be combined with treatment with an anticancer agent either associated with the microbubble, co-administered locally with the microbubble, or administered to the patient by any suitable delivery route and dosage regimen in conjunction with, but not necessarily simultaneously, with the microbubble and nitro-fatty acids.

Percutaneous localized delivery of the microbubbles may be accomplished, for example and without limitation, using a catheter or cannula, including trocars and hypodermic needles. Percutaneous delivery can be controlled by use of a medical syringe, or a pump such as an infusion pump or a peristaltic pump. A multitude of catheter designs are described in the medical arts and are available commercially. A catheter can be a thin tube that is inserted, e.g., into a patient's vasculature. A catheter may be guided to reach a specific internal location, and can be directed, often using a guide wire, to a specific location within the patient's vasculature, for example and without limitation, to a coronary artery for delivery to a site of a thrombus or MVO. The catheter may be an infusion catheter. The catheter may be a catheter used for thrombolytic procedures, which may be a tube, typically a thin, hollow plastic flexible tube that has one or more openings in its distal end through which a therapeutic composition can be passed. Liquids, such as a composition comprising the microbubbles described herein, can be passed through the catheter for localized delivery within a patient's vasculature. The catheter may be a balloon catheter. Balloon catheters comprise one or two distal expandable elements (balloons) that seal a blood vessel, thereby facilitating localization of any composition delivered through the catheter. The catheter may be an infusion catheter combined with one or more ultrasonic transducers, such as the EKOS Ultrasound Guided Catheter system commercially available from Boston Scientific.

For treatment of a patient, the microbubble can be administered by any effective route of administration, such as, without limitation, by: parenteral administration, such as by intravenous, intraarterial, intraperitoneal, intra-organ, such as delivery to the heart, or at the site of a clot or MVO, intratumoral injection, intralesional injection, intradermal injection, subdermal injection, or intramuscular injection. The composition may be administered as an individual dose, a continuous dose, or in multiple doses over time.

Also provided herein are pharmaceutical compositions and formulations which can include the microbubbles and nitro fatty acid described herein. In one aspect, provided herein are pharmaceutical compositions containing the microbubbles and nitro fatty acid, as described herein, and a pharmaceutically acceptable carrier. The pharmaceutical compositions containing the microbubbles and nitro fatty acid are useful in the methods described herein. Such pharmaceutical compositions can be formulated based on the mode of delivery. One example is compositions that are formulated for systemic administration via parenteral delivery, e.g., by local delivery via a catheter, cannula, or hypodermic needle. The pharmaceutical compositions may be administered in dosages sufficient for, e.g., thrombolysis or MVO ablation, and/or reduction of inflammation. A suitable dose of the microbubbles and nitro fatty acid is, for example and without limitation from 3 to 6 milligrams, from 3 to 5 milligrams, or from 4 to 6 milligrams. Certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the microbubbles and nitro fatty acid encompassed herein can be made using conventional methodologies, or based upon in vivo testing using an appropriate animal model.

### EXAMPLES

Previous strategies at treating or preventing MVO included administration of vasodilators and antiplatelet therapy, thrombus aspiration and embolic protection devices, and even the use of hyperoxemic intracoronary reperfusion therapy (Rezkalla, S. H. et al. Management of No-Reflow Phenomenon in the Catheterization Laboratory. JACC Cardiovasc Interv., 2017, 10:215-223). However, even with these various strategies, no definitive therapeutic consensus for MVO has arisen, and a number of clinical trials have yielded conflicting results (Jaffe, R. et al. Prevention and treatment of microvascular obstruction-related myocardial injury and coronary no-reflow following percutaneous coronary intervention: a systematic approach, JACC Cardiovasc Interv., 2010, 3:695-704; Niccoli, G. et al. Myocardial no-reflow in humans, J Am Coll Cardiol, 2009, 54:281-92; Mazhar, J. et al. Predictors and outcome of no-reflow post primary percutaneous coronary intervention for ST elevation myocardial infarction, Int J Cardiol Heart Vasc., 2016, 10:8-12). Therefore, there is a need for an acute therapy capable of resolving MVO and its inflammatory sequelae due to the unpredictable and rapidly catastrophic nature of acute cardiac events.

In this regard, nitro-fatty acids (NFAs) are endogenous signaling molecules formed from oxidative reactions between NO and nitrite-derived species and unsaturated fatty acids (Woodcock, S. R. et al. Nitrated fatty acids: synthesis and measurement, Free Radic Biol Med., 2013, 59:14-26). These molecules have been previously investigated in a wide range of inflammatory conditions due to their role in post-translational modification of various anti-inflammatory signaling pathways (Rubbo, H. Nitro-fatty acids: novel anti-inflammatory lipid mediators. Braz J Med Biol Res., 2013, 46:728-34; Villacorta, L et al. Nitro-fatty acids in cardiovascular regulation and diseases: characteristics and molecular mechanisms, Frontiers in bioscience (Landmark edition), 2016, 21:873; Rudolph, V. et al. Endogenous generation and protective effects of nitro-fatty acids in a murine model of focal cardiac ischaemia and reperfusion. Cardiovasc Res, 2010, 85:155-66; Cui, T. et al. Nitrated fatty acids: Endogenous anti-inflammatory signaling mediators. J Biol Chem., 2006, 281:35686-35698; Freeman, B. A. et al. Nitro-fatty acid formation and signaling. J Biol Chem., 2008, 283:15515-15519; Kansanen, E. et al. Nrf2-dependent and -independent responses to nitro-fatty acids in human endothelial cells: identification of heat shock response as the major pathway activated by nitro-oleic acid. J Biol Chem., 2009, 284:33233-33241; Baker, P. R. et al. Fatty acid transduction of nitric oxide signaling: multiple nitrated unsaturated fatty acid derivatives exist in human blood and urine and serve as endogenous peroxisome proliferator-activated receptor ligands. J Biol Chem., 2005, 280:42464-42475; Schopfer, F. J. et al. Nitrolinoleic acid: an endogenous peroxisome proliferator-activated receptor gamma ligand. Proc Natl Acad Sci USA., 2005, 102:2340-2345), resulting in decreased production of reactive oxide species, inhibition of pathogenic macrophage conversion and activation, inhibition of fibrotic and hyperplastic processes, and prevention of endothelial dysfunction (Cole, M. P. et al. Nitro-fatty acid inhibition of neointima formation after endoluminal vessel injury, Circ Res., 2009, 105:965-972; Koudelka, A. et al. Nitro-Oleic Acid Prevents Hypoxia- and Asymmetric Dimethylarginine-Induced Pulmonary Endothelial Dysfunction, Cardiovasc Drugs Ther., 2016, 30:579-586; Ambrozova, G. et al. Nitro-oleic acid modulates classical and regulatory activation of macrophages and their involvement in pro-fibrotic responses, Free Radic Biol Med., 2016, 90:252-260; Rudolph, T. K. et al. Nitrated fatty acids suppress angiotensin II-mediated fibrotic remodelling and atrial fibrillation, Cardiovasc Res., 2016, 109:174-184). All of these effects are clinically relevant to the prevention and treatment of MVO, especially as NFAs have been demonstrated to be cardioprotective in the setting of focal cardiac ischemia (V. Rudolph et al., Cardiovasc Res, 2010; Nadtochiy, S. M. et al. Nitroalkenes confer acute cardioprotection via adenine nucleotide translocase 1, J Biol Chem., 2012, 287:3573-3580).

Use of therapeutic ultrasound for thrombolysis has become a rising subject of investigation. The efficacy of ultrasound-targeted microbubble cavitation (UTMC) in the treatment of MVO and improvement of tissue perfusion has been investigated previously (Pacella, J. J. et al. Treatment of microvascular micro-embolization using microbubbles and long-tone-burst ultrasound: an in vivo study. Ultrasound in Medicine and Biology, 2015, 41:456-464; Yu, F. T. et al. The Role of Nitric Oxide during Sonoreperfusion of Microvascular Obstruction, Theranostics, 2017, 7:3527). This technique involves image-guided ultrasound sonication of intravenously injected lipid microbubble (MB) contrast agents, resulting in microbubble oscillation (cavitation) which generates intravascular shear forces. UTMC produces both mechanical and biological effects in disrupting microthrombi to restore perfusion and activating endothelial nitric oxide (NO) pathways for local vasodilation (Yu et al., 2017; Leeman, J. E. et al. Effect of acoustic conditions on microbubble-mediated microvascular sonothrombolysis, Ultrasound in Medicine and Biology, 2012, 38:1589-1598; Chen, X. et al. New insights into mechanisms of sonothrombolysis using ultra-high-speed imaging. Ultrasound in Medicine and Biology, 2014, 40:258-262). In addition to thrombolysis, UTMC may also be used as a means for targeted delivery of therapeutics through the use of loaded microbubbles, resulting in site-specific concentration of the therapeutic at higher efficiency than through systemic administration, while also reducing off-target effects (Carson, A. R. et al. Gene therapy of carcinoma using ultrasound-targeted microbubble destruction, Ultrasound in Medicine and Biology, 2011, 37:393-402; Carson, A. R. et al. Ultrasound-targeted microbubble destruction to deliver siRNA cancer therapy, Cancer Research, 2012, 72(23): 6191-6199; Kopechek, J. A. et al. Ultrasound targeted microbubble destruction-mediated delivery of a transcription factor decoy inhibits STAT3 signaling and tumor growth. Theraostics, 2015, 5:1378; Kopechek, J. A. et al. Cardiac gene expression knockdown using small inhibitory RNA-loaded microbubbles and ultrasound, PLoS One, 2016, 11(7):e0159751; Yu, F. T. et al. Low Intensity Ultrasound Mediated Liposomal Doxorubicin Delivery Using Polymer Microbubbles. Mol Pharm., 2016, 13:55-64). UTMC is an ideal solution for MVO because it is a minimally invasive, theranostic technique that may visualize regions of MVO using microbubbles asultrasoundcontrast agents while simultaneously delivering image-guided ultrasound pulses.

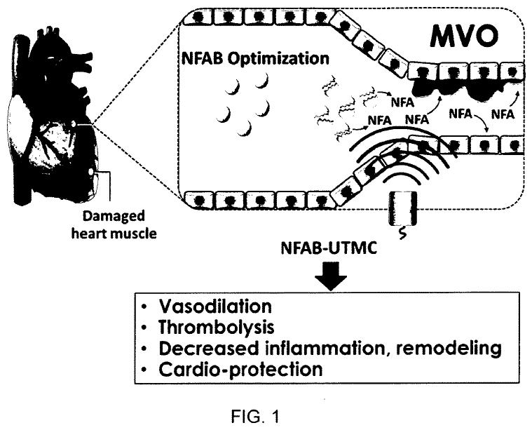

Incorporation of the lipophilic NFA into the lipid shell of a microbubble for use with UTMC may allow for targeted local delivery of NFA and subsequent anti-inflammatory and cardioprotective effects to be focused at the specific location of MVO while supplementing the mechanical effects of UTMC on thrombolysis and perfusion (FIG. 1). Site specific oscillation of NFA-loaded microbubbles and release of NFA within the microcirculation at the target site may create a region of concentrated NFA.

### Example 1: Synthesis of Nitro-Fatty Acid Microbubbles

Nitro-fatty acid microbubbles (NFABs) were synthesized with a combination of 1,2-distearoyl-sn-glycero-3-phosphocholine (DSPC), 1,2-distearoyl-sn-glycero-3-phosphoethanolamine-N-(methoxy(polyethylene glycol)-2000] (ammonium salt) (DSPE-mPEG), polyoxyethylene (40) stearate (ST-PEG(40)), and a nitro-fatty acid (NFA) species, such as 9-nitro-oleic acid, in chloroform.

Stock solutions of DSPC, DSPE-mPEG, and ST-PEG(40), having a concentration of 25 milligrams per milliliter (mg/mL) each, were prepared. A stock solution of 10 mg/mL NFA in chloroform was prepared. Ten mg of ST-PEG(40) was added to the 20 mL glass vial (400 μL of the 25 mg/mL stock solution). Fifteen mg (20 mg when NFA is not included) of DSPC was added to the 20 mL glass vial (400 μL of the 25 mg/mL stock solution; 600 μL of the 25 mg/mL solution to obtain 20 mg). Ten mg of DSPE-mPEG 2000 was added to the 20 mL glass vial (400 μL of the 25 mg/mL stock solution). Five mg, 1 mg, 0.5 mg, or 0.25 mg of NFA (such as 9-nitro-oleic acid) in chloroform (500 μL of the 10 mg/mL stock solution for 5 mg) was added to a 20 milliliter (mL) glass vial. The combined materials in the glass vial were dried under argon for 25 minutes, until the vial was mostly dried. The 20 mL glass vial was placed under vacuum overnight, where the glass vial was vented with two 18 gauge needles and the cap vial loosened. Following the vacuum, the vial was checked for an oily residue.

A saline bag was filled approximately 70% full with perfluorobutane gas, followed by the addition of 15 mL of acidic saline. Four mL of saline, using a micropipette, was added to the dried lipid solution in the 20 mL vial. An ultrasonic cleaner, in sonic mode, was used to detach the dried lipid from the sides of the vial. The vial was swirled vigorously on the ultrasound focal point in a water bath until no undissolved lipid solids remained in the vial. The dissolved lipid solution was sonicated using a sonicator at level 3.5 for 30 seconds, where the sonicator tip was placed close to the bottom of the vial, but not touching the bottom of the vial. The contents of the vial were sonicated for 30 seconds and then the vial was submerged in an ice water bath. This sonication-cooling process was repeated 10 times. The solution became translucent when the lipid was fully dissolved in the solution. The vial containing the translucent solution was placed on ice until cooled.

To obtain Sample 0, 10 μL of the lipid solution was withdrawn from the vial and placed in 200 μL of acetonitrile.

The vial containing the lipid solution was placed into the vacuum bag with the vial lid loosened. The air was removed from the bag using a vacuum and was replaced with perfluorobutane. A low flow of perfluorobutane was added to the bag such that positive pressure was maintained.

The lipid solution was sonicated using sonicator with the 75 second program on level 5, while the bag was full of perfluorobutane. The sonicator tip was placed 2 millimeters below the surface of the liquid. In the last 15 seconds of the sonicator's 75 second program, the vial was tilted and rotated such that the sonicator probe reached all the edges. This process formed the microbubble suspension. The lid was placed on the vial and removed from the vacuum bag.

Approximately 10 mL of saline was added to the microbubble suspension. Using an 18 gauge needle and 10 mL syringe, 10 mL of the microbubble suspension was transferred to the saline bag full of perfluorobutane and acidic saline. The addition of 10 mL of saline and the solution transfer to the saline bag process was repeated two times. Following the last addition of the microbubble suspension into the saline bag, the bag was left to rest for 60 minutes.

After 60 minutes, the subnatant (approximately 20 mL) was removed and the microbubble layer on top was left undisturbed. Twenty mL of saline was added to the bag and the contents of the bag were gently mixed. The contents of the bag were left to rest for 60 minutes. After 60 minutes, 20 mL of the subnatant was removed and the microbubble layer on the top was left undisturbed. Two mL, or more as needed, was added to the bag. All of the remaining liquid was removed from the bag using a 5 mL syringe. If necessary, additional saline was added to reach a total volume 5 mL.

An appropriate number of 2 mL screw top rubber seal vials were filled with saline using two 25 gauge needles and a syringe. The saline was replaced with perfluorobutane gas by venting upside down. No saline remained in the seal vials.

The microbubble suspension was aliquoted into the perfluorobutane filled 2 mL rubber seal vials. Each vial contained approximately 0.5 mL of microbubble suspension.

The vials of microbubble suspensions were stored in the refrigerator (2° C. to 8° C.) for up to 2 weeks.

### Example 2: Design and Testing of Nitro-Fatty Acid Microbubble for the Treatment of Ischemia-Reperfusion Injury Using Ultrasound-Targeted Microbubble Cavitation

### Materials and Methods

### Nitro-Fatty Acid Microbubble Synthesis and Characterization

Nitro-fatty acid microbubbles (NFABs) were synthesized with a combination of DSPC (Avanti Polar Lipids Inc., Alabaster, Ala.), DSPE-mPEG (Avanti Polar Lipids), ST-PEG(40) (Sigma Aldrich, St. Louis, Mo.), and 9-nitro-oleic acid as the nitro-fatty acid (NFA). DSPC, DSPE-mPEG, ST-PEG(40), and 9-nitro-oleic acid were combined in a glass vial with chloroform in a 29.1:5.2:43.8:21.9 molar ratio, respectively, before the resulting solution was vaporized under an argon gas stream before vacuum storage. The dehydrated lipids were then rehydrated in acidic saline with a head of perfluorobutane gas (FluoroMed, LP, Round Rock, Tex., USA) prior to sonication (XL2020, Qsonica LCC, Neton, Conn.). The resulting NFABs were then washed three times in acidic saline to remove unsonicated lipids from solution before being aliquoted into sealed glass vials containing perfluorobutane, as described above in Example 1.

The NFABs were characterized for microbubble concentration, size distribution, and loading efficiency of NFA. All measurements were taken serially up to 6 days post-synthesis to allow for the tracking of stability in storage and loss of NFA content over time. Concentration and size distribution were measured using a Coulter Counter (Multisizer 3 Beckman Coulter, Fullerton, Calif., USA). NFA content of the final NFAB solution and the solution subnatant (for quantification of free NFA) was measured using liquid chromatography/mass spectrometry. Free NFA was subtracted from the overall solution NFA measurement resulting in a concentration of only NFA loaded onto NFABs.

As a comparison to the NFABs, in-house lipid microbubbles were synthesized using the same lipid components as above, but in the absence of NFA, using the same procedures for synthesis and quantification of size and concentration. For contrast-enhanced ultrasound imaging, DEFINITY® (Lantheus Medical Imaging, North Billerica, Mass.) was used.

### Measurement of NFA in NFABs and Tissue Samples

For samples of NFABs, both the microbubbles in saline solution and samples of the saline subnatant alone were measured for NFA content. Subnatant NFA concentration was subtracted from overall microbubble solution NFA concentration to give the concentration of incorporated NFA only.

For tissue samples, skeletal muscle tissue was taken from the treatment site (gastrocnemius muscle) and a control site (quadriceps femoris muscle) on the ipsilateral hindlimb. The differential of NFA between the two sites was obtained by subtraction of their respective NFA concentrations. The protocol for NFA concentration quantification using liquid chromatography/mass spectrometry is documented in previous literature (V. Rudolph et al., Cardiovasc Res, 2010).

### High Speed Microscopy

High-speed microscopic imaging was used to visualize the dynamic behavior of the NFABs during ultrasound excitation using the ultrafast microscopy system at the University of Pittsburgh Medical Center (Chen, X. et al. Ultra-fast bright field and fluorescence imaging of the dynamics of micrometer-sized objects, Review of Scientific Instruments, 2013, 84:063701). The system is capable of recording microscopic movies at up to 25 million frames-per-second for 128 frames.

### Animal Preparation

The animal studies performed in this investigation received prior approval by the Institutional Animal Care and Use Committee at the University of Pittsburgh. The rat hindlimb model previously developed in our laboratory was used for modeling of both healthy microvasculature and microvasculature experiencing ischemia-reperfusion injury (Yu, G. Z. et al. Ultrasound-Targeted Microbubble Cavitation with Sodium Nitrite Synergistically Enhances Nitric Oxide Production and Microvascular Perfusion, Ultrasound Med Biol., 2020, 46:667-678).

Briefly, anesthesia was induced using 2.5% inhaled isoflurane in Sprague-Dawley rats (Charles Rivers, Pittsburgh, Pa., USA) weighing 275±20 grams (g). The right femoral artery and right internal jugular vein were then cannulated for intravascular access. The distal right femoral artery was ligated, and the infusion line was advanced to the level of the iliac bifurcation for direct access to the left femoral artery. The jugular access was used to deliver DEFINITY® for contrast-enhanced ultrasound imaging undiluted at a rate of 2 milliliters per hour (mL/hr). The left femoral access was used to deliver treatment microbubbles and therapeutic agents. Treatment groups for the experiments included free NFA alone with a vehicle of 85:15 polyethylene glycol to ethanol (Free NFA only), ultrasound-targeted microbubble cavitation (UTMC) using unmodified treatment microbubbles with co-infusion of free NFA (Free NFA+UTMC), and UTMC with NFAB infusion only (NFAB+UTMC).

### Ultrasound Targeted Cavitation Therapy and Perfusion Imaging

For the UTMC treatment, therapeutic ultrasound was administered using a single-element transducer operating at 1 MHz frequency (A3035, 0.5 inch, Olympus NDT, Waltham, Mass.), driven with an arbitrary function generator (AFG3252, Tektronix, Beaverton, Oreg.) and a radio frequency power amplifier (800A3B, Amplifier Research, Souderton, Pa.). The ultrasound system was calibrated with a 200-μm capsule hydrophone (HGL-0200, Onda Corp, Sunnyvale, Calif., USA). The treatment transducer was fixed vertically above the gastrocnemius region of the left rat hindlimb with the rat placed in right lateral decubitus position at a distance of approximately 1.5 centimeters (cm) from the hindlimb and was coupled to the hindlimb using acoustic gel.

The therapeutic ultrasound pulse (center frequency 1 MHz, peak negative acoustic pressure 1.5 MPa, pulse duration 5 millisecond (ms) was delivered with 3 second pulse interval to allow new microbubble to reach the treatment area for 10 minutes for each treatment session. Each treatment session was followed by contrast-enhanced ultrasound imaging using a clinical imaging probe (Siemens, Sequoia, 15L8 probe) and infusion of DEFINITY®. Microbubbles were infused with automated syringe pumps and the syringes were rotated during administration to maintain microbubbles in suspension.

Contrast-enhanced ultrasound imaging was performed using the burst-replenishment technique in contrast pulse sequence mode at 7 MHz frequency with a mechanical index of 0.2 and framerate of 5 frames/second. The imaging ultrasound transducer was positioned horizontally, parallel to the longitudinal axis of the rat tibia and perpendicular to the treatment transducer. The imaging transducer was manipulated such that the junction of the saphenous and popliteal arteries was visible in the field of view and the skin thickness was minimized. Occasional artifacts, potentially representing bone reflections, were permitted but ultimately excluded from any regions of interest for analysis. A 5-frame burst with mechanical index of 1.9 was used to extinguish contrast microbubbles from the field of view. Both the initial burst and subsequent reperfusion of the hindlimb with contrast microbubbles were recorded for further processing and analysis of hemodynamic parameters. All other ultrasound parameters including dynamic range (60 decibel (dB)), gain (0 dB), and compression curve (linear) were kept constant throughout the studies for all treatment groups.

### Image Processing

Processing of all recorded cine-loops from burst-replenishment imaging was performed using in-house code in MATLAB as described previously (Yu et al., 2020). Briefly, microvasculature regions of interest were selected for the gastrocnemius microvasculature such that skin, bone, and major vessels (saphenous and popliteal) were excluded. Image brightness (representing 60 dB range) of the region of interest was analyzed as a function of time for each cine-loop (approx. 30 seconds) and fit to the following exponential regression:

y(t)=A(1−e^{-βt}), Equation 1

where A is the plateau value β is the inverse time constant. The product A*β is the initial slope of the curve (accounting for the steepness of the increase of the brightness to the plateau value over time). Hemodynamically, the plateau value A was taken to represent the microvascular blood volume while the product A*β was taken to represent the microvascular blood flow rate.

### Healthy Hindlimb Model Experimental Protocol

Treatment groups for the healthy hindlimb model were centered around analysis of tissue delivery of the loaded NFA and included free NFA alone, NFA with co-infusion of unmodified MB UTMC, and UTMC with NFABs. Dosage of NFA in all treatment groups was kept constant. The actual dose was equal to the amount of NFA loaded onto 1×10⁹ NFABs. Similarly, all treatment microbubbles (regardless of NFA loading) were kept constant (1×10⁹ microbubbles per treatment). All treatment infusions (NFA or MB infusions) were administered via the femoral cannulation at 3 mL/hr. All imaging microbubble infusions were administered via the internal jugular cannulation at 2 mL/hr.

Placement of the therapeutic transducer over the gastrocnemius muscle was confirmed by visual inspection and one pulse of microbubble destruction in the field of view prior to treatment. After each 10-minute treatment, infusion of imaging microbubbles was performed during burst-replenishment imaging and cine-loop recording. Rat physiologic parameters, including heart rate, respiratory rate, and oxygen saturation, were continuously monitored. After the second 10-minute treatment, the rat was euthanized by isoflurane overdose followed by heart excision. Following euthanasia, tissue samples were obtained from the UTMC-treated gastrocnemius muscle tissue as guided by ultrasound imaging markers. Tissue samples were also collected from the ipsilateral quadriceps femoris muscle as a control for the UTMC treatment receiving the same systemic perfusion of infused agents without targeted ultrasound stimulation. All tissue samples were then immediately frozen in liquid nitrogen and stored at −80 degrees Celsius (° C.).

### Ischemia-Reperfusion Injury Model Experimental Protocol

Aside from the addition of ischemia-reperfusion injury itself, the general protocol for the rat hindlimb model was identical to that of the healthy hindlimb model. Prior to treatment, the left femoral artery was ligated using a suture, resulting in isolated left hindlimb ischemia. After an hour of ischemia, the suture was removed, allowing for reperfusion of the hindlimb for half an hour. After this half hour, treatment proceeded as with the healthy hindlimb model. After the second 10-minute treatment and perfusion imaging, there was a 2-hour wait to allow for development of pathophysiologic changes in gene expression before the animal was sacrificed and tissue samples were collected as previously detailed.

### Determination of Oxidative Stress

Malondialdehyde (MDA), measured as a thiobarbituric acid reactive substance (TBARS), is a secondary product of lipid peroxidation. Skeletal muscle tissue samples were homogenized in ice-cold buffer and centrifuged at 12,000 g for 15 minutes at 4° C. After centrifugation, supernatants were processed as per the manufacturer's instruction of a TBARS Assay Kit (Cayman Chemical Company, Ann Arbor, Mich., USA). The absorbance of the supernatant was measured at 532 nm. Thiobarbituric acid reacts with MDA to form a TBARS di-adduct, a pink chromogen detectable at 532 nm. Results were expressed as MDA (ε=1.56×10⁵ reciprocal Molar reciprocal cm (M⁻¹cm⁻¹)) per milligram of protein measured using a standard Bradford assay (Thermo Fisher Scientific, Waltham, Mass., USA).

### Gene Expression Analysis—Real Time Quantitative PCR

The skeletal muscle tissue samples from the treatment and control groups previously described were homogenized in the presence of TRIzol™ reagent (ThermoFischer Scientific, Waltham, Mass., USA) using an Ultrasonic Processor (Heat Systems, XL-2020). Chloroform (200 μL) was added to 1 milliliter (mL) of TRIzol™ solution, centrifuged at 12,000 g for 15 minutes at 4° C., and 200 μL of aqueous upper layer was pipetted into an RNAse-free centrifuge tube. After subsequent treatment with isopropyl alcohol and 75% ethanol, the RNA pellet was dissolved in diethyl pyrocarbonate (DEPC) water. Complementary DNA was prepared from 2 micrograms (μg) of total RNA using TaqMan reverse transcription kit (Applied Biosystems, Foster City, Calif., USA). RT-PCR amplifications were performed with the SYBR™ Green PCR Master Mix (Thermo Scientific, Waltham, Mass., USA) using a CFX96 Connect™ Real-Time PCR Detection System (Bio-Rad, USA). Target genes included intercellular adhesion molecule 1 (ICAM-1), vascular cell adhesion molecule 1 (VCAM-1), monocyte chemotactic protein 1 (MCP-1), tumor necrosis factor alpha (TNF-α), interleukin-6 (IL-6), and nuclear factor kappa-light-chain-enhancer of activated B cells (NF-κB). The relative mRNA levels of target genes were normalized to GAPDH mRNA. The primer sequence used for RT-PCR analysis includes:

| Name | Sequence |
|---|---|
| TNF-α Forward (SEQ ID NO: 1) | 5′-CTCTTCTCATTCCCGCTCGT-3′ |
| TNF-α Reverse (SEQ ID NO: 2) | 5′-GGGAGCCCATTTGGGAACTT-3′ |
| IL-6 Forward (SEQ ID NO: 3) | 5′-CCAGTTGCCTTCTTGGGACT-3 |
| IL-6 Reverse (SEQ ID NO: 4) | 5′-TCTGACAGTGCATCATCGCT-3′ |
| ICAM-1 Forward (SEQ ID NO: 5) | 5′-CAAACGGGAGATGAATGGT-3′ |
| ICAM-1 Reverse (SEQ ID NO: 6) | 5′-TCTGGCGGTAATAGGTGTAAA-3′ |
| MCP-1 Forward (SEQ ID NO: 7) | 5′-CTGACCCCAATAAGGAATG-3′ |
| MCP-1 Reverse (SEQ ID NO: 8) | 5′-TGAGGTGGTTGTGGAAAAGA-3′ |
| VCAM-1 Forward (SEQ ID NO: 9) | 5′-TTTGCAAGAAAAGCCAACATGAAAG-3′ |
| VCAM-1 Reverse (SEQ ID NO: 10) | 5′-TCTCCAACAGTTCAGACGTTAGC-3′ |
| NF-κB Forward (SEQ ID NO: 11) | 5′-ACGATCTGTTTCCCCTCATCT-3′ |
| NF-κB Reverse (SEQ ID NO: 12) | 5′-TGGGTGCGTCTTAGTGGTATC-3′ |
| GAPDH Forward (SEQ ID NO: 13) | 5′-AAACCCATCACCATCTTCCA-3′ |
| GAPDH Reverse (SEQ ID NO: 14) | 5′-GTGGTTCACACCCATCACAA-3′ |

### Statistical Analysis

All statistical analyses were performed in Prism (GraphPad Prism version 8.00, GraphPad Software, La Jolla, Calif., USA). Analyses were performed as either one-way analysis of variances (ANOVAs) or two-way repeated measures ANOVAs when appropriate. Post-hoc analyses for two-way repeated measures ANOVAs were performed using Sidak's multiple comparisons test, while post-hoc for one-way ANOVAs used Tukey's multiple comparisons test. Significance was taken at p<0.05. All error bars shown indicate standard deviation.

### Results

### Nitro-Fatty Acid Microbubble Characterization

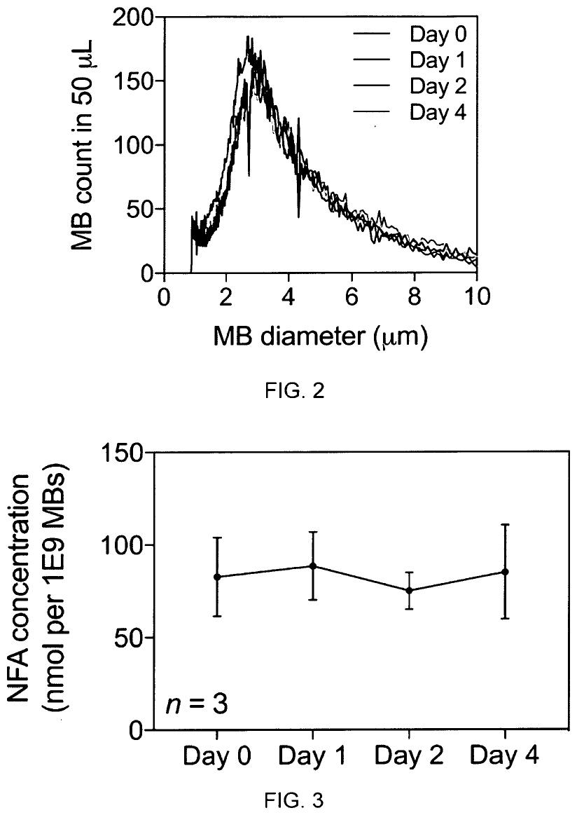

Size and distribution of NFABs were measured from Coulter counter analysis (FIG. 2) and include results from a representative batch up to 4 days post-synthesis. Quantitatively there were no significant differences in mean and standard deviation of NFAB diameter (3.0±1.5 μm) over the 4 days measured, with NFAB concentration dropping only to 85% of the original concentration from day 1 onward. The distribution curves depict a unimodal population roughly centered at the mean similar to that observed in previous lipid microbubble Coulter counter measurements.

Loaded NFA content for three batches of NFABs were serially measured up to 4 days post-synthesis in triplicate with results shown in FIG. 3. The average concentration was approximately 82.9±5.8 nanomoles (nmol) of NFA per 1×10⁹ microbubbles after subtraction of the subnatant unloaded NFA content. Because the therapeutic dose for microbubbles previously established in the rat hindlimb model is 1×10⁹ microbubbles delivered, the loaded NFA content of 1×10⁹ microbubbles was taken as the equivalent dosage for the free NFA only and free NFA with control MB UTMC groups.

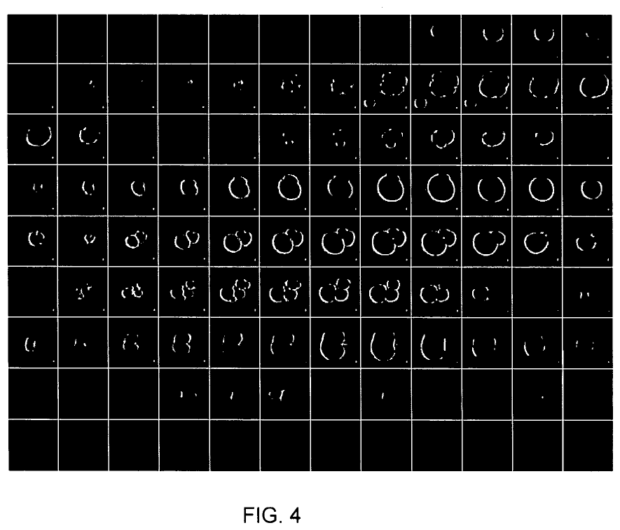

High-speed microscopy during ultrasound stimulation of NFABs revealed that these microbubbles behaved similarly as other lipid-based microbubbles. Consecutive still frame images of a high-speed movie is shown in FIG. 4 (1 MHz, 1.5 MPa). Since a negative peak acoustic pressure of 1.5 MPa is well above the inertial cavitation threshold, these images demonstrate several morphological changes. The destruction of the microbubbles and loss of spherical structure are immediately noticeable. Daughter microbubbles formation is also observed.

### Healthy Hindlimb Model

### Perfusion Changes in Contrast-Enhanced Ultrasound Imaging

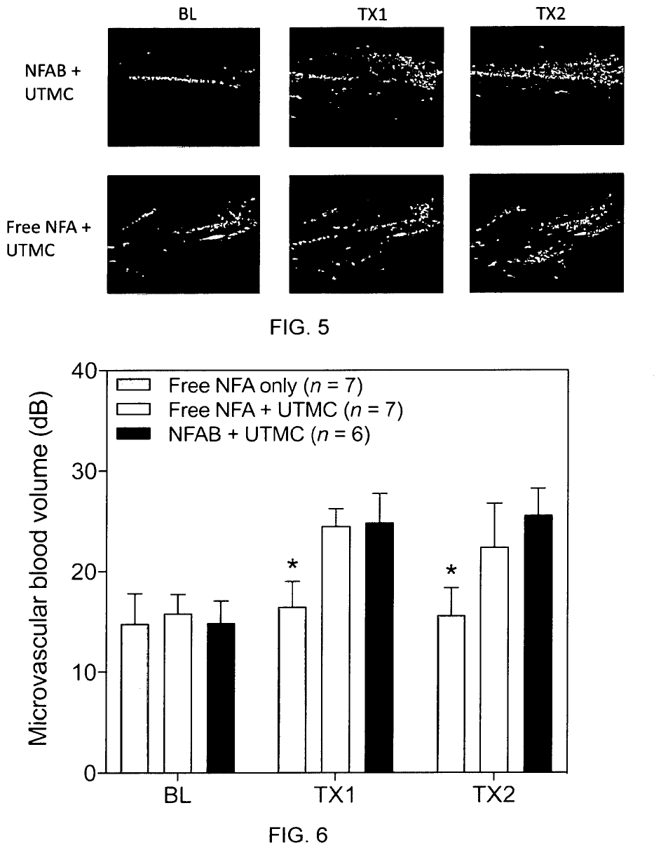

Burst-replenishment imaging representative still-frames at 5 seconds post-burst for the two UTMC-treated healthy hindlimb groups are shown in FIG. 5. As the main difference between these two groups is the compartment of incorporation of NFA into the microbubble structure versus co-infusion with unmodified microbubbles, differences between these groups was taken to represent the effect of incorporating NFA into the lipid microbubble shell.

The NFAB group showed not only a greater area coverage of contrast-visualized reperfusion as well as a greater image intensity compared to the baseline state. The free NFA co-infusion group with regular UTMC does not show greater intensity compared to baseline, as well as incomplete reperfusion at 5 seconds post-burst. Although this is not representative of a persistent hypoperfused region given that at 30 seconds post-burst, the hypointense region was successfully reperfused, this abridged comparison further emphasizes the improved speed of microvascular reperfusion seen in the NFAB treatment group. Still-frame visualization demonstrated increased speed of microvascular perfusion of the NFAB group that was further reinforced by quantification of microvascular perfusion using region-of-interest selection.

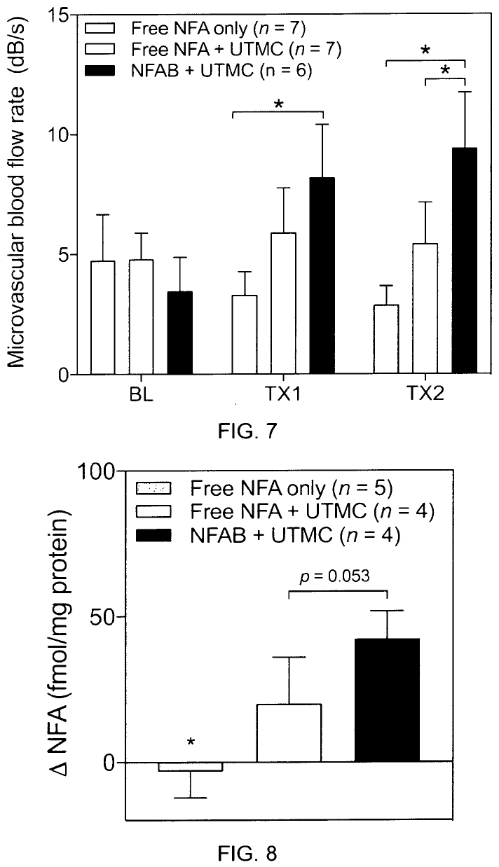

Quantification of the hindlimb cine-loops using an exponential regression for region-of-interest image intensity is presented in FIGS. 6 and 7. Although both the NFAB and free NFA co-infusion with UTMC groups showed increases in microvascular blood volume, only the NFAB group demonstrated increased microvascular flow rate as well. As a control, free NFA alone did not affect hindlimb microvascular perfusion. These results suggest that while UTMC itself had a significant effect on increasing blood volume, the incorporation of NFA into the microbubble shells offered additional hemodynamic benefits.

### Tissue Delivery of NFA

Tissue concentrations of NFA at both ultrasound treated and untreated sites for the three groups are shown in FIG. 8 as differences in concentration between the two sites. Free NFA alone showed no difference in concentration (as no UTMC was applied to the hindlimb in this group), while NFA co-infusion with regular UTMC and NFAB+UTMC showed sequentially increasing differences in concentration. Incorporation of NFA into the lipid microbubble resulted in approximately twice the increase in tissue delivery of NFA compared to NFA administration as a co-infusion with control microbubbles.

### Ischemia-Reperfusion Injury Model

### Perfusion Changes in Contrast-Enhanced Ultrasound Imaging

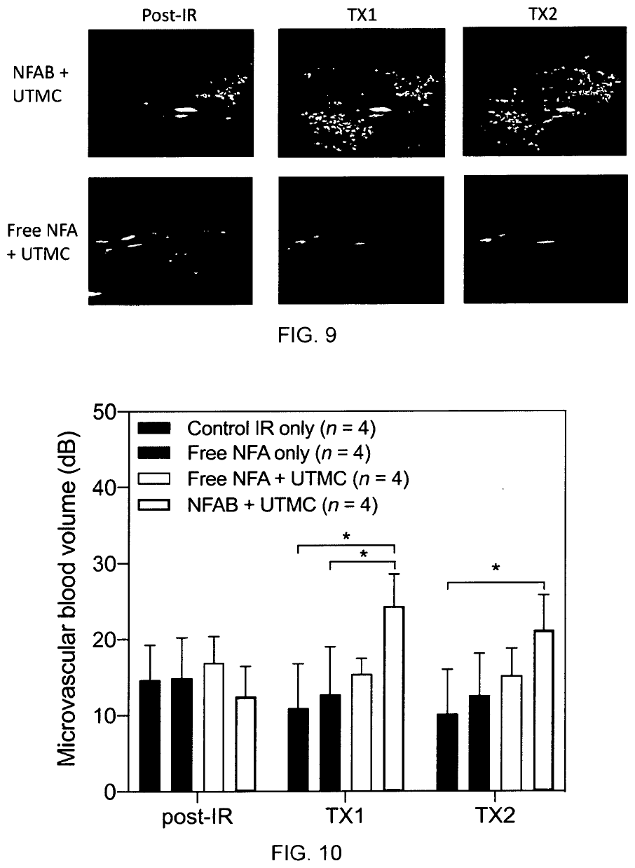

Still-frames from the burst-replenishment cine-loops are shown in FIG. 9 for the NFAB and NFA co-infusion with standard UTMC groups. The NFAB group showed significant increases in perfusion compared to the post-ischemia-reperfusion state while the NFA co-infusion group showed no improvements with potentially even decreases in perfusion.

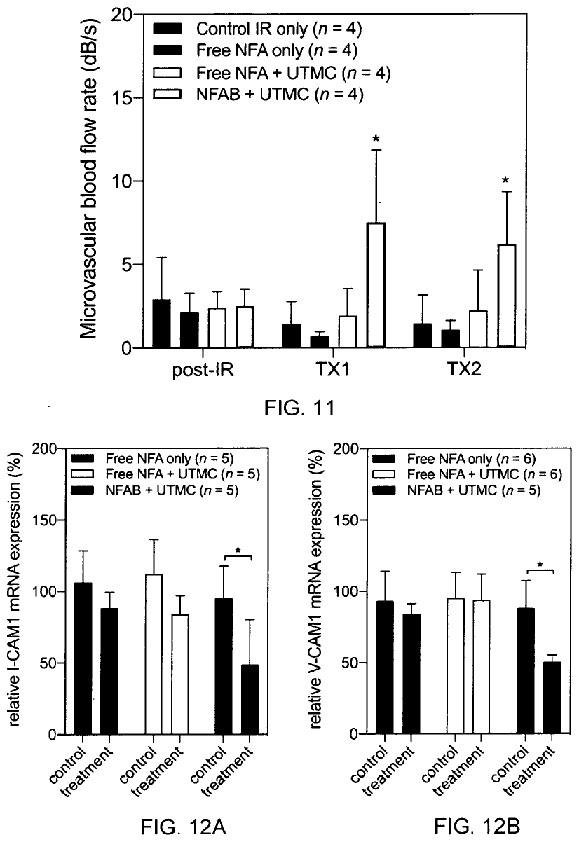

Quantitative analysis of hindlimb perfusion showed that only the NFAB group had significant increases in microvascular blood volume as well as microvascular blood flow after each treatment (FIGS. 10 and 11). In addition, the free NFA only group and the NFA co-infusion group showed decreased mean microvascular blood flow after each treatment below the flow rate immediately after ischemia-reperfusion injury.

### UTMC-Mediated NFA Delivery Downregulates Target Genes in Hindlimb Model In Vivo

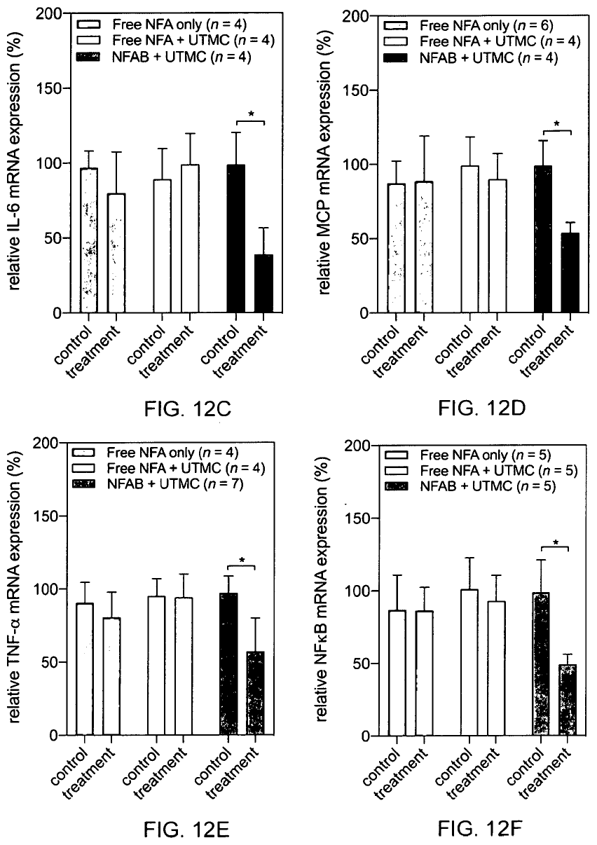

Results of gene expression analysis for all target genes are shown in FIGS. 12A-12F. For I-CAM1 (FIG. 12A), V-CAM1 (FIG. 12B), MCP (FIG. 12D), and TNF-α (FIG. 12E), only the NFAB group resulted in significantly lower gene expression in the treatment sample compared to the control sample. In addition, the treated NFAB sample showed a lower level of gene expression than treated samples from all other groups. Although the NFA only and NFA co-infusion groups showed lower mean relative treatment group I-CAM1 expression compared to their respective control groups, these differences were not statistically significant.

NFAB UTMC Reduces Oxidative Stress after Ischemia-Reperfusion Injury

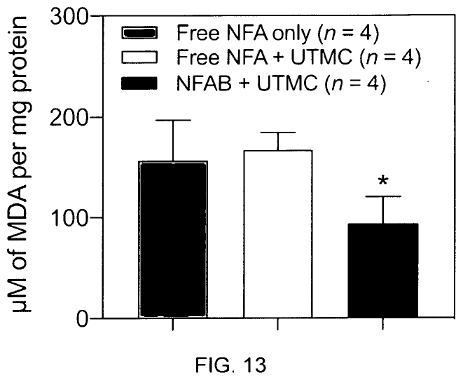

Measurements of MDA per mg of protein for the three groups are shown in FIG. 13. The NFAB+UTMC group demonstrated significantly lower concentration of MDA compared to both free NFA only and free NFA+UTMC groups, which were not significantly different between themselves.

NFA-conjugated lipid MB (where the lipid shell is actually constructed with a lipophilic NFA, 9-nitro-oleic acid), in the presence of image-guided therapeutic ultrasound, resulted in targeted delivery of NFA, as measured by increased local tissue delivery of NFA compared to both free NFA alone and free NFA with UTMC using control microbubbles. Application of UTMC with NFAB significantly increased microvascular blood flow, suppressed inflammatory cytokine transcription, and reduced oxidative stress in an ischemia-reperfusion injury model.

NFAs present potential therapeutic interactions with UTMC from their capacity to increase nitric oxide (NO) bioavailability through multiple mechanisms. These include direct dissociation of NO molecules from the parent NFA molecule through the Nef chemical reaction, which then mediate cGMP-dependent signaling pathways (Schopfer, F. J. et al. Fatty acid transduction of nitric oxide signaling Nitrolinoleic acid is a hydrophobically stabilized nitric oxide donor, The Journal of Biological Chemistry, 2005, 280:19289-19297). In addition, NFAs have been shown to increase eNOS mRNA and protein expression as well as serine-1177 phosphorylation, resulting in increased cellular distribution and activity of eNOS (Khoo, N. K. et al. Activation of vascular endothelial nitric oxide synthase and heme oxygenase-1 expression by electrophilic nitro-fatty acids, Free Radic Biol Med., 2010, 48:230-239). The molecular structure of NFAs is also similar to the phospholipid species found in the lipid shell of certain therapeutic microbubbles used in UTMC. Another advantage of delivering NFA through loaded microbubbles in UTMC is the ability to concentrate payloads of NFA at the specific site of MVO. Previous literature has shown that the inhibition of various inflammatory signaling reactions by NFAs are both time-dependent and dosage-dependent in inflammatory and endothelial cells (Villacorta, L. et al. Nitro-fatty acids in cardiovascular regulation and diseases: characteristics and molecular mechanisms. Frontiers in bioscience (Landmark edition), 2016, 21:873). This, coupled with previous successes of reducing inflammation and preserving myocardium in a myocardial ischemia model with systemically administered NFA, suggests the strong potential of further improving the efficacy of this therapy through rapidly concentrating NFAs at the site of the myocardial insult. This further complements the rapid-onset and localized nature of microvascular obstruction (MVO).

### NFAB UTMC Provides a Means for Rapid Targeted NFA Delivery

NFAs as therapeutic agents have generally demonstrated significant anti-inflammatory effects in previous studies of tissue ischemic injury and atherosclerosis. However, in application to acutely developing, localized pathologies such as acute myocardial infarction (AMI) and subsequent MVO, systemic delivery of NFAs is insufficient. To this end, while previous studies of NFAs have featured non-specific, systemic a priori delivery, we demonstrate that UTMC focally enhances tissue delivery of NFA in a relatively rapid manner. Two ten-minute UTMC treatments resulted in a significant increase in local skeletal muscle concentration of NFA over adjacent untreated sites. Although NFAs do not have adverse systemic side effects that would otherwise limit their therapeutic feasibility, enhancing focal delivery remains desirable for acute, localized pathologies.

### NFABs Retain Typical Dynamic Microbubble Behavior

Using ultra-high-speed microscopy of the NFABs during sonication revealed that they undergo similar acoustic behaviors as previously noted typical of lipid microbubbles, including successful inertial cavitation as well as the formation of smaller daughter microbubbles, which are thought to be significant to the therapeutic efficacy of lipid microbubbles in UTMC therapy (Chen, X. et al. Dynamic behavior of microbubbles during long ultrasound tone-burst excitation: Mechanistic insights into ultrasound-microbubble mediated therapeutics using high-speed imaging and cavitation detection, Ultrasound in Medicine and Biology, 2016, 42:528-538). We have shown that these properties are relevant and associated with mechanical “chiseling” of microthrombi to ultimately relieve the physical obstruction associated with MVO (Chen et al., 2016). In designing the novel NFAB described herein, retention of these properties was a relevant consideration for the NFAB construct.

### NFA Cause Downregulation of Inflammatory Gene Expression

The NFAB+UTMC treatment, which provides targeted delivery of NFA, resulted in a significant reduction in the expression of numerous inflammatory target genes ubiquitously expressed during ischemia-reperfusion injury, a major contributor to the sequelae of MVO. Specifically, TNF-α is a pro-inflammatory factor secreted by monocytes and vascular endothelial cells in ischemia-reperfusion injury which promotes nuclear translocation of NF-κB. This results in expression of inflammatory cytokines including IL-6 and MCP-1, as well as adhesion molecules on the surface of vascular endothelial cells such as VCAM-1 and ICAM-1. This cascade is predictive of not only MVO but also future cardiovascular events, as the resulting inflammation produces increased oxidative stress and tissue damage, ultimately worsening patient outcomes (see, e.g., Yellon, D. M. et al. Myocardial reperfusion injury, New England Journal of Medicine, 2007, 357:1121-1135).

Our results showed concurrent decreases in transcription of all of these inflammatory factors with NFAB+UTMC treatment that were not seen in systemic administration of free NFA alone or free NFA with UTMC. It is unclear at present whether the efficacy of NFAB+UTMC was due to temporally accelerated delivery of NFA to the treatment site or due to lowered therapeutic dosage requirements of NFA given the enhanced local delivery efficiency, or a combination of these factors. These mechanisms may be further investigated with additional time intervals assessing for treatment response of equivalent systemic administration, as well as additional concentrations of NFA.

UTMC with NFAB Results in Increased Perfusion

Microvascular hemodynamic changes were also assessed in this study through contrast-enhancedultrasound imaging. Although we have previously shown that microvascular perfusion may be enhanced with UTMC therapy in combination with nitrite, a similar effect was not expected for the combination of NFAB+UTMC. This is because while nitrite is a known reservoir of nitric oxide bioavailability and has several identified mechanisms of chemical conversion to nitric oxide especially in environments of hypoxia and acidosis, NFAs have been noted to only directly serve as a nitric oxide donor in strongly aqueous environments that are highly distinct from the biological intravascular compartment (Duranski, M. R. et al. Cytoprotective effects of nitrite during in vivo ischemia-reperfusion of the heart and liver, J Clin Invest. 2005, 115:1232-1240; Lundberg, J. O. et al. The nitrate-nitrite-nitric oxide pathway in physiology and therapeutics, Nat Rev Drug Discov., 2008, 7:156-167). Therefore, the mechanism of enhanced perfusion by NFAs is unlikely to result from direct donation of nitric oxide. This is also supported by our results showing that infusion of free NFA alone had no significant effects on hindlimb perfusion. Interestingly, free NFA with UTMC also did not result in enhanced microvascular perfusion. That said, the combination of NFA with UTMC with microbubbles remains a promising option.

In conclusion, NFABs were developed and demonstrated the therapeutic potential of NFAB+UTMC treatment in an ischemia-reperfusion injury model. In doing so, a rapid, targeted treatment for suppression of inflammatory gene expression and protection against oxidative stress has been developed. This therapy is especially suitable for treatment of localized sequelae of AMI, including MVO and ischemia-reperfusion injury, as well as other inflammatory vascular pathologies. The value of bioactive lipids using UTMC as a vehicle for targeted delivery, with opportunities for synergy with the other effects of UTMC therapy including enhanced microvascular perfusion and mechanical disruption of thrombi has also been demonstrated.

### Example 3: Exemplary Ischemia-Reperfusion Injury Clinical Trial

Treatment of localized sequelae of acute myocardial infarction, including MVO and ischemia-reperfusion injury, as well as other inflammatory vascular pathologies using UTMC therapy in combination with NFABs could relieve the physical obstruction in the microcirculation and target the associated inflammatory milieu.

Objective: As a primary outcome, the treatment of ischemia-reperfusion injury in a patient will be determined after using UTMC to deliver NFA released from injected NFABs at the site of injury.

Study Design: A randomized, controlled, double-blinded, single-center trial of NFABs will be intravenously administered to patients having an ischemia-reperfusion injury. UTMC will be used to deliver to the NFAs contained within the NFA to the site of the ischemia-reperfusion injury. Preliminary results in rats show NFABs delivered to the site of ischemia-reperfusion injury showed significant increases in perfusion at the site of injury, as compared to the post-ischemia reperfusion injury state.

### Example 4

Murine model studies of electrophilic nitroalkene actions on tissue responses to Ang II, hypoxia and obesity showed nitro-oleic or nitro-linoleic acid inhibit fibrotic vascular and tissue remodeling in heart, liver and lung. In aggregate, nitroalkene administration will induce a reduction of α-smooth muscle actin (αSMA), connexin 43 and collagen deposition, suppression of macrophage activation, inhibition of mitochondrial oxidant generation, activation of caspase-dependent apoptosis and inhibition of SMAD/TGFβ signaling. These are all events linked with clinical liver, cardiac and pulmonary fibrosis

The molecular pharmacology of the two pleiotropically-acting drugs approved specifically for fibrosis therapy, pirfenidone and nintedanib, overlaps with that of small molecule nitroalkenes. Pirfenidone has an electrophilic α,β-unsaturated ketone substituent and, like nitroalkenes, activates Nrf2-dependent gene expression and inhibits oxidative stress, NFkB and MAPK dependent signaling(Liu, Y., Lu, F., Kang, L., Wang, Z., and Wang, Y. (2017) Pirfenidone attenuates bleomycin-induced pulmonary fibrosis in mice by regulating Nrf2/Bach1 equilibrium. BMC pulmonary medicine 17, 63; Li, Z., Liu, X., Wang, B., Nie, Y., Wen, J., Wang, Q., and Gu, C. (2017) Pirfenidone suppresses MAPK signalling pathway to reverse epithelial-mesenchymal transition and renal fibrosis. Nephrology (Carlton) 22, 589-597; and Nakanishi, H., Kaibori, M., Teshima, S., Yoshida, H., Kwon, A. H., Kamiyama, Y., Nishizawa, M., Ito, S., and Okumura, T. (2004) Pirfenidone inhibits the induction of iNOS stimulated by interleukin-1beta at a step of NF-kappaB DNA binding in hepatocytes. J Hepatol 41, 730-736). Nintedanib is a competitive inhibitor of the ATP-binding pocket in the related PDGF, FGF and VEGF receptor tyrosine kinases and suppresses downstream tyrosine kinase signaling (Ahmad, K., and Nathan, S. D. (2018) Novel management strategies for idiopathic pulmonary fibrosis. Expert review of respiratory medicine 12, 831-842 and Lederer, D. J., and Martinez, F. J. (2018) Idiopathic Pulmonary Fibrosis. N Engl J Med 379, 797-798), as do nitroalkenes by different mechanisms (Freeman, B. A., Pekarova, M., Rubbo, H., and Trostchansky, A. (2017) Electrophilic Nitro-Fatty Acids: Nitric Oxide and Nitrite-Derived Metabolic and Inflammatory Signaling Mediators. Nitric Oxide Biology and Pathology, 3rd Edition, 213-229, 213-229).

Nitroalkenes can reverse established fibrosis. Bleomycin-treated 3 mo old C57Bl6 mice (n=7), showed a significant reversal of fibrosis following 2 additional weeks of 7.5 mg/kg oral NO₂-oleic acid. Both Masson's trichrome and αSMA staining showed the resolution of pathologic remodeling. Ashcroft scoring revealed decreased collagen deposition. qRT-PCR of TGFβ, Col1A1, αSMA, TGFβ, vimentin (mesenchymal genes), MMP7 and E-cadherin expression showed NO₂-OA inhibition of these pro-fibrotic indices.

Further, inhibition of hepatic fibrosis was seen in a model of long-term NASH diet-induced liver damage (Rom, O., Xu, G., Guo, Y., Zhu, Y., Wang, H., Zhang, J., Fan, Y., Liang, W., Lu, H., Liu, Y., Aviram, M., Liu, Z., Kim, S., Liu, W., Wang, X., Chen, Y. E., and Villacorta, L. (2019) Nitro-fatty acids protect against steatosis and fibrosis during development of nonalcoholic fatty liver disease in mice. EBioMedicine 41, 62-72). Non-invasive imaging using photoacoustic-ultrasound established steatosis and fibrosis were diagnosed prior experimental therapy with NO₂-OA. Controls included equimolar amounts oleic acid (OA) and vehicle. CLAMS and NMR-based analysis demonstrates that OA-NO₂ improved body composition and energy metabolism, while inhibiting hepatic triglyceride (TG) accumulation. Photoacoustic-ultrasound imaging revealed a robust inhibition of liver steatosis and fibrosis by NO₂-OA. RNA-sequencing analysis of liver tissue revealed inflammation and fibrosis as major pathways suppressed by NO₂-OA administration, as well as regulation of lipogenesis and lipolysis pathways. There was also inhibition of SREBPI proteolytic activation and subsequent lipogenesis gene expression by NO₂-OA. These results were reinforced by histological analysis and quantification of lipid accumulation, lobular inflammation (F4/80 staining), fibrosis (collagen deposition, αSMA staining) and liver damage (plasma ALT). Parallel in vitro studies indicated that NO₂-OA inhibited TG biosynthesis and accumulation in hepatocytes and inhibited fibrogenesis in human stellate cells.

Consequently, administration of NFA-containing or NFA-ester-containing microbubbles as described herein and co-administration of microbubbles and an NFA or NFA ester, to a site of fibrosis or fibrogenesis in a patient, e.g., by injection or via catheter delivery, followed by disruption of the microbubbles with ultrasound as described herein, is expected to treat the fibrosis, reduce production of a fibrotic lesion, or reduce a fibrotic lesion in the patient.

### Example 5

As described above, local reduction of inflammation at or within a tumor or precancerous lesion is expected to be useful in treatment of cancer or a precancerous lesion in a patient. Consequently, administration of NFA-containing or NFA ester-containing microbubbles as described herein and co-administration of microbubbles and NFAs or NFA esters, to a site of a tumor or a precancerous lesion in a patient, e.g., by injection or via catheter delivery, followed by disruption of the microbubbles with ultrasound as described herein, is expected to reduce inflammation at the site of the tumor or precancerous lesion, thereby treating the cancer or precancerous lesion in the patient.

Having described this invention above, it will be understood to those of ordinary skill in the art that the same can be performed within a wide and equivalent range of conditions, formulations, and other parameters without affecting the scope of the invention or any embodiment thereof. Any document incorporated herein by reference is only done so to the extent of its technical disclosure and to the extent it is consistent with the present document and the disclosure provided herein.

1. A microbubble, comprising: a lipid-based shell comprising a circumferential region that defines a core of the microbubble, a gas within the core of the microbubble, and a nitro-fatty acid or an ester thereof, or a pharmaceutically-acceptable salt of the nitro-fatty acid or ester thereof.

2. The microbubble of claim 1, wherein the nitro-fatty acid or ester thereof, or a pharmaceutically-acceptable salt of the nitro-fatty acid or ester thereof is incorporated into the exterior circumferential region of the lipid-based shell.

3. The microbubble of claim 1, wherein the nitro-fatty acid or ester thereof, or a pharmaceutically-acceptable salt of the nitro-fatty acid or ester thereof is contained within the core of the microbubble.

4. The microbubble of claim 1, wherein the gas comprises air, oxygen, nitrogen, argon, or a perfluorocarbon.

5. The microbubble of claim 1, wherein the perfluorocarbon is perfluorobutane.

6. The microbubble of claim 1, wherein the lipid based shell comprises a phospholipid, a phosphatidylcholine, a glycerol-phosphoethanolamine lipid that is optionally PEGylated, and/or a PEGylated fatty acid.

7-10. (canceled)

11. The microbubble of claim 6, wherein the lipid-based shell comprises: 1,2-distearoyl-sn-glycero-3-phosphocholine; 1,2-Distearoyl-sn-Glycero-3-Phosphoethanolamine-N-[Methoxy(Polyethylene glycol)-2000]; and PEG-stearate, e.g. polyoxyethylene (40) stearate.

12. The microbubble of claim 1, where the nitro-fatty acid or ester thereof, or pharmaceutically-acceptable salt of the nitro-fatty acid or ester thereof, is associated with the lipid-based shell in a ratio as compared to other lipids present in the lipid shell ranging from 10 to 25% by mass or from 20 to 40% by moles.

13. (canceled)

14. The microbubble of claim 1, comprising: 9-nitrooleic acid,
10-nitrooleic acid, 9-nitrolinoleic acid, 10-nitrolinoleic acid, 12-nitrolinoleic acid,
13-nitrolinoleic acid, 9-Nitrononanoic acid, 9-nitro-9-trans-octadecenoic acid, 10-nitro-9-trans-octadecenoic acid, or a salt or ester thereof.

15. The microbubble of claim 1, comprising a nitro-fatty acid having the structure: 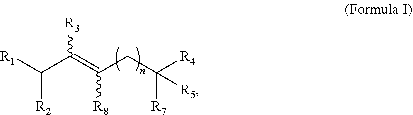
wherein R₁ is hydrogen, C₁-C₂₄ alkyl or C₁-C₂₄ alkenyl; R₂, R₃, R₇, and R₈ are each independently, hydrogen, oxygen, C₁-C₂₄ alkyl or NO₂; R₄ is a terminal COOR₆ group, wherein R₆ is hydrogen, C₁-C₂₄ alkyl, or a pharmaceutically acceptable counterion; R₅ is hydrogen, C₁-C₂₄ alkyl, or R₄ and R₅ collectively form ═C(R₉)(R₁₀), wherein R₉ comprises C₁-C₂₄ alkyl or C₁-C₂₄ alkenyl, or wherein R₉ is a terminal COOR₆ group, and R₁₀ is hydrogen or NO₂; n is from 1 to 24; and wherein at least one of R₂, R₃, R₇, R₈, and R₁₀, when present, is an NO₂ group; 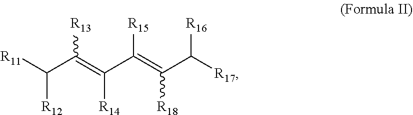
wherein R₁₁ is hydrogen, C₁-C₂₄ alkyl, or C₁-C₂₄ alkenyl; R₁₂, R₁₄, R₁₅ and R₁₆ are hydrogen, C₁-C₂₄ alkyl, or C₁-C₂₄ alkenyl; R₁₃ and R₁₈ are each independently, hydrogen, oxygen, C₁-C₂₄ alkyl, or NO₂; R₁₇ is a terminal COOR₁₉ group, wherein R₁₉ is hydrogen, C₁-C₂₄ alkyl, or a pharmaceutically acceptable counterion, wherein at least one of R₁₃, R₁₄, R₁₅, and R₁₈ is NO₂; or 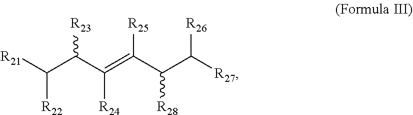
wherein R₂₁ is hydrogen, C₁-C₂₄ alkyl, or C₁-C₂₄ alkenyl; R₂₂, R₂₄, R_{25′} and R₂₆ are hydrogen; one of R₂₃ and R₂₈ is NO₂, and the other of R₂₃ and R₂₈ is ONO₂; and R₂₇ is a terminal COOR₂₉ group, wherein R₂₉ is hydrogen, C₁-C₂₄ alkyl, or a pharmaceutically acceptable counterion,
or an ester thereof, or a pharmaceutically-acceptable salt of the nitro-fatty acid or ester thereof.

16. A therapeutic composition, comprising: a microbubble as claimed in claim 1; and a pharmaceutically acceptable excipient, one or more additional therapeutically active agents or visualization agents, or any combinations thereof.

17. A unit dosage form comprising the therapeutic composition of claim 16 in a medical syringe or a reservoir configured to connect to a catheter.

18. A method of treating localized inflammation in a patient, comprising delivering the composition of claim 16, to a site of localized inflammation in the patient and administering an ultrasound pulse to the site effective to deliver the nitro-fatty acid or ester thereof, or the pharmaceutically-acceptable salt of the nitro-fatty acid or ester thereof, to the site or to disrupt the microbubble, thereby reducing inflammation at the site.

19. The method of claim 18, wherein the site of localized inflammation is associated with a thrombus or a microvascular obstruction and the ultrasound pulse is effective for thrombolysis of the thrombus or the microvascular obstruction, or the site of localized inflammation is associated with fibrosis or cancer, a site of a stent, an aneurism coil, or a valve replacement, a site of a cardiovascular surgical procedure, a site of a pulmonary embolism, a site of a fibrotic lesion, or a site of a tumor or precancerous lesion.

20-25. (canceled)

26. A method of clot thrombolysis in a patient, comprising delivering the composition of claim 16 to a site of a thrombus or microvascular obstruction in a patient, and administering ultrasound effective for thrombolysis of the thrombus or the microvascular obstruction and to release the nitro-fatty acid or ester thereof, or the pharmaceutically-acceptable salt of the nitro-fatty acid or ester thereof, at the site.

27. The method of claim 26, wherein the site of the thrombus or microvascular obstruction in the patient is in the patient's heart, optionally in or adjacent to a myocardial infarct, thrombus, or microvascular obstruction.

28. (canceled)

29. A method of treating a fibrotic lesion in a patient, comprising delivering the composition of claim 16 to a site of a fibrotic lesion in a patient, and administering ultrasound effective to release the nitro-fatty acid at the site, thereby reducing the size of fibrotic lesion and/or preventing further fibrosis.

30. A method of reducing inflammation in a patient, comprising delivering a gas-filled microbubble and a nitro-fatty acid or an ester thereof, or a pharmaceutically-acceptable salt of the nitro-fatty acid or ester thereof, to a site of localized inflammation in a patient and administering an ultrasound pulse to the site effective to disrupt the microbubbles, thereby reducing inflammation at the site.

31. The method of claim 30, wherein the site of localized inflammation is associated with a thrombus or a microvascular obstruction and the ultrasound pulse is effective for thrombolysis of the thrombus or the microvascular obstruction.

32. The method of claim 30, wherein the site of localized inflammation is associated with fibrosis or cancer, is a site of a stent, an aneurism coil, or a valve replacement, is a site of a cardiovascular surgical procedure, is a site of pulmonary embolism, is a site of a fibrotic lesion, or is a site of a tumor or precancerous lesion.

33-38. (canceled)
